(19) United States
(12) Patent Application Publication
(10) Pub. No.: US 2026/0042719 A1
(43) Pub. Date: Feb. 12, 2026

(54) SYSTEM AND METHOD FOR RECOVERING NAPHTHALENE FROM A RERUN COLUMN BOTTOM SAMPLE

(71) Applicant: HPCL-Mittal Energy Limited, Bathinda (IN)

(72) Inventors: Hemant Tyagi, Meerut (IN); Upendra Kumar Mall, Mau (IN); Neeraj Kumar, Ludhiana (IN); Ramdev Kushwaha, Rewa (IN)

(21) Appl. No.: 19/365,708

(22) Filed: Oct. 22, 2025

(30) Foreign Application Priority Data
Jun. 19, 2025 (IN) 202511058764

(51) Int. Cl. C07C 7/14; C07C 7/00; C07C 7/04; C07C 7/10; C07C 7/144

(52) U.S. Cl. CPC C07C 7/14; C07C 7/005; C07C 7/04; C07C 7/10; C07C 7/144; C07B 2200/13

(57) ABSTRACT

A method for recovering high-purity naphthalene from a Rerun Column bottom residue is disclosed. The process begins with collecting the residue and dividing it into two portions. One portion is maintained at room temperature, while the second is cooled to approximately 16° C. to initiate the selective solidification of naphthalene. Upon gradual warming of the cooled portion to ambient temperature over 3-4 hours, a clear separation between solid naphthalene and the liquid residue is achieved. The liquid layer is decanted, and the remaining solid fraction is filtered using Whatman-1 filter paper with tissue paper to absorb residual liquids. The recovered crystals are washed 3-4 times with water to eliminate impurities and dried in a hot air oven at 100±2° C. for 2 hours. Final settling at room temperature yields dry, white crystalline naphthalene with a melting point of 80.2° C., a purity of 97.27%, and a recovery efficiency of 77.7%.

### CROSS-REFERENCE TO RELATED APPLICATION

This application claims the benefit of and priority to India (IN) Patent Application No. 202511058764 filed Jun. 19, 2025, the contents of which being incorporated by reference in their entirety herein.

### TECHNICAL FIELD

The present disclosure relates generally to chemical processing and separation technologies, and more particularly to a system and method for recovering naphthalene from a chemical process stream, such as a rerun column bottom sample.

### BACKGROUND

Naphthalene, a bicyclic aromatic hydrocarbon with the chemical formula C₁₀H₈ and a molecular weight of 128.17 g/mol, is a crucial industrial compound. It is characterized by its relatively low melting point (80° C.) and higher boiling point (218° C.). While insoluble in water, Naphthalene readily dissolves in organic solvents such as benzene, ether, and chloroform. A notable property is its tendency to sublime easily at room temperature, transitioning directly from solid to vapor without melting. Despite being highly flammable and burning with a sooty flame, Naphthalene's diverse applications underscore its significant commercial value.

The utility of Naphthalene extends across numerous industries, serving as a vital chemical intermediate. Although historically known for its use in mothballs and pest repellents due to its toxic vapor, its more substantial applications lie in its role as a precursor for phthalic anhydride, a key component in plasticizers, dyes, and resins. It is also extensively employed in the manufacturing of azo dyes and synthetic tanning agents for the dye and pigment industry. Furthermore, Naphthalene derivatives find specific, important uses in military explosives, jet fuel additives, and certain cleaning products for odor control. In the polymer industry, naphthalene sulfonates are crucial as dispersants in concrete, dyes, and detergents, highlighting the compound's broad and indispensable role.

Despite the high demand and value, current industrial processes often lead to significant Naphthalene losses in waste streams, particularly the “Rerun Column Bottom”. Analysis of this stream consistently reveals a substantial Naphthalene content, typically ranging from 9.3% to 26.2%, a variation dependent on specific plant operating conditions. The composition of the Rerun Column Bottom includes a density of 965.0-980.1 kg/m3 (average: 970.9 kg/m3), an Initial Boiling Point (IBP) of 171.4-190.8° C. (average: 185.3° C.), and a Final Boiling Point (FBP) of 218.6-250.5° C. (average: 239.6° C.). Other characteristics include an existent gum content of 44-98 mg/100 ml (average: 75 mg/100 ml), a flash point of 65.0-72.0° C. (average: 69.0° C.), a kinematic viscosity of 1.67 cSt at 50° C., and a moisture content of 60 mg/kg. The elevated Naphthalene content in this stream is primarily due to the current operation of the Gasoline Fractionator (GF) Overhead (0/H) temperature at approximately 105° C., which is higher than the targeted 92° C.

The financial and environmental implications of this loss are considerable. Considering a minimum Naphthalene content of 9.3% and a typical Rerun Column Bottom production rate of 10-12 tons per hour, approximately 1 ton of Naphthalene is lost hourly, amounting to roughly 24 tons per day. This substantial daily loss represents a significant economic burden and an environmental concern, as a valuable resource is being discarded. Therefore, there is a clear and urgent need for an efficient and economically viable method to recover Naphthalene from this waste stream. Such an invention would not only mitigate financial losses but also foster more sustainable industrial practices by maximizing resource utilization, leading to increased production efficiency, reduced waste, and a positive impact on the overall profitability for Naphthalene producers.

### BRIEF SUMMARY

The present disclosure seeks to provide a system and method for the efficient recovery of naphthalene from industrial chemical process streams, specifically targeting streams like the rerun column bottom. This disclosure addresses the significant economic and environmental losses associated with the current discard of valuable naphthalene in these waste streams. The system and method aim to extract and purify naphthalene that is currently present in substantial quantities (typically 9.3% to 26.2%) within streams such as the rerun column bottom. By recovering this valuable chemical, the invention seeks to enhance overall process efficiency, reduce waste, and generate additional revenue from a previously discarded resource, thereby contributing to both economic gain and environmental sustainability.

In an embodiment, a system for recovering naphthalene from a rerun column bottom sample is disclosed. The system includes an extraction unit configured to receive 100 ml of rerun column bottom sample and 100 ml of methanol, and mix them via agitation for 2 minutes.

The system further includes a phase separation chamber connected to the extraction unit to allow the mixture to rest undisturbed for 5 to 6 hours to achieve clear separation of methanol and raffinate phases.

The system further includes a separation mechanism coupled to the phase separation chamber to isolate the methanol-rich upper phase from the raffinate lower phase.

The system further includes a multi-stage extraction arrangement wherein the extraction unit and separation chamber are used iteratively with fresh methanol for multiple cycles.

The system further includes an evaporation unit configured to heat the combined methanol-rich extracts at approximately 70° C. to evaporate methanol and isolate a crystalline naphthalene residue.

The system further includes a purification unit connected to the evaporation unit comprising a series of washing chambers for sequentially washing the crystalline residue with dilute sulphuric acid, caustic solution, and water.

The system further includes a drying and melting chamber coupled to the purification unit to heat the washed residue to approximately 100° C. to remove moisture and convert the naphthalene into a liquid state.

The system further includes a cooling and crystallization unit in continuation with the drying and melting chamber to allow the liquid naphthalene to cool to approximately 25° C. to obtain solid naphthalene crystals.

In another embodiment, a method for recovering naphthalene from a rerun column bottom sample is disclosed. The method includes collecting at least one sample of Rerun Column bottom residue.

The method further includes subjecting a first portion of the sample to ambient room temperature of approximately 25° C. and a second portion to a reduced temperature of approximately 16° C.

The method further includes observing the solidification of Naphthalene in the second portion maintained at 16° C.

The method further includes allowing the second portion to gradually equilibrate to room temperature over a period of 3-4 hours until a clear separation between the solid Naphthalene layer and liquid residue is formed.

The method further includes decanting the upper liquid layer from the partially solidified sample.

The method further includes filtering the remaining portion using Whatman-1 filter paper and placing tissue paper beneath the filter to assist in absorbing residual liquid traces.

The method further includes washing the recovered solid crystals with water 3-4 times to remove impurities.

The method further includes subjecting the washed crystals to drying in a hot air oven operated at approximately 100±2° C. for about 2 hours.

The method further includes removing the beaker from the oven and allowing any remaining liquid to settle at room temperature.

The method further includes obtaining dry white crystalline Naphthalene with a melting point of approximately 80.2° C., a purity of about 97.27%, and a recovery efficiency of approximately 77.7%.

An object of the present disclosure is to provide a highly effective and selective method for the recovery of Naphthalene from complex rerun column bottom streams.

Another object of the present disclosure is to achieve high-purity Naphthalene, specifically aiming for a crude Naphthalene product with a purity exceeding 97%.

Another object of the present disclosure is to enable the further refinement of recovered Naphthalene to the level of refined Naphthalene through additional purification steps.

Another object of the present disclosure is to offer a scalable and industrially viable process for Naphthalene recovery that can be efficiently implemented.

Another object of the present disclosure is to minimize waste and enhance resource utilization by recovering valuable Naphthalene from a previously challenging stream.

Yet another object of the present invention is to deliver an expeditious and cost-effective robust and reliable system capable of precisely controlling the critical parameters, such as cooling temperature (≈16° C.), settling conditions (≈25° C.), and washing/drying steps.

To further clarify the advantages and features of the present disclosure, a more particular description of the invention will be rendered by reference to specific embodiments thereof, which are illustrated in the appended drawings. It is appreciated that these drawings depict only typical embodiments of the invention and are therefore not to be considered limiting of its scope. The invention will be described and explained with additional specificity and detail in the accompanying drawings.

### BRIEF DESCRIPTION OF FIGURES

These and other features, aspects, and advantages of the present disclosure will become better understood when the following detailed description is read concerning the accompanying drawings in which like characters represent like parts throughout the drawings, wherein:

FIG. 1 illustrates a block diagram of a system for recovering naphthalene from a rerun column bottom sample in accordance with an embodiment of the present disclosure;

FIG. 2 illustrates a flow chart of a method for recovering naphthalene from a rerun column bottom sample in accordance with an embodiment of the present disclosure;

FIG. 3 illustrates exemplary profiles of recovered Naphthalene in accordance with an embodiment of the present disclosure;

FIG. 4(a) illustrates exemplary Pictorial representation of samples for a first cooling and decanting process for recovery of Naphthalene in accordance with an embodiment of the present disclosure;

FIG. 4(b) illustrates exemplary clear layer of solid Naphthalene for a first cooling and decanting process for recovery of Naphthalene in accordance with an embodiment of the present disclosure;

FIG. 4(c) illustrates exemplary Picture of recovered Naphthalene for a first cooling and decanting process for recovery of Naphthalene in accordance with an embodiment of the present disclosure;

FIG. 5(a) illustrates exemplary Pictorial representation of samples for a second cooling and decanting process for recovery of Naphthalene in accordance with an embodiment of the present disclosure;

FIG. 5(b) illustrates exemplary clear layer of solid Naphthalene for a second cooling and decanting process for recovery of Naphthalene in accordance with an embodiment of the present disclosure;

FIG. 5(c) illustrates exemplary Picture of recovered Naphthalene for a second cooling and decanting process for recovery of Naphthalene in accordance with an embodiment of the present disclosure;

FIG. 6 illustrates Chromatogram Naphthalene Standard in accordance with an embodiment of the present disclosure;

FIG. 7 illustrates Chromatogram against Experiment 1 in accordance with an embodiment of the present disclosure;

FIG. 8 illustrates Chromatogram against Experiment 2 in accordance with an embodiment of the present disclosure;

FIG. 9 illustrates Chromatogram against Experiment 3 in accordance with an embodiment of the present disclosure; and

FIG. 10 illustrates a processes for recovery of Naphthalene in accordance with an embodiment of the present disclosure.

Further, skilled artisans will appreciate those elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

### DETAILED DESCRIPTION

To promote an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components. Unless otherwise defined, all technical and scientific terms used herein have the same meaning as commonly understood by one of ordinary skill in the art to which this invention belongs. The system, methods, and examples provided herein are illustrative only and not intended to be limiting. Embodiments of the present disclosure will be described below in detail concerning the accompanying drawings.

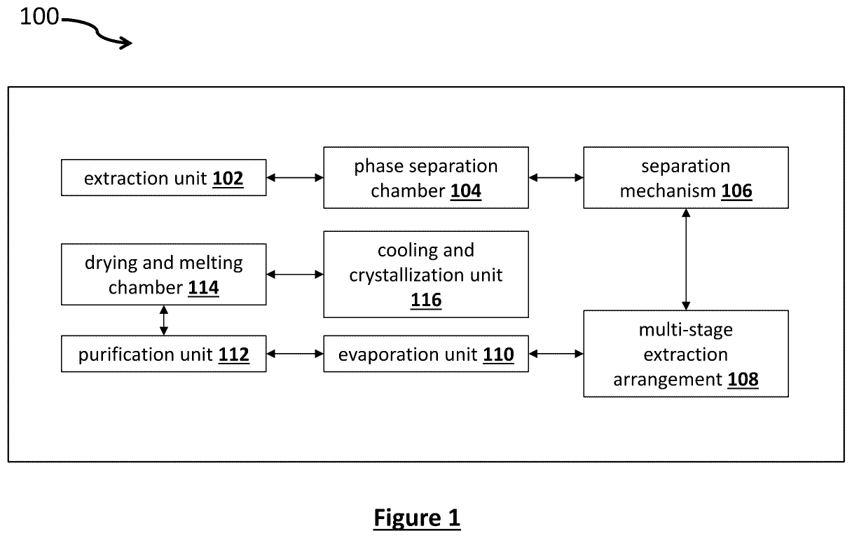

Referring to FIG. 1, a block diagram of a system for recovering naphthalene from a rerun column bottom sample is illustrated in accordance with an embodiment of the present disclosure. The system 100 includes an extraction unit 102 configured to receive 100 ml of rerun column bottom sample and 100 ml of methanol, and mix them via agitation for 2 minutes.

In an embodiment, a phase separation chamber 104 is connected to the extraction unit 102 to allow the mixture to rest undisturbed for 5 to 6 hours to achieve clear separation of methanol and raffinate phases.

In an embodiment, a separation mechanism 106 is coupled to the phase separation chamber 104 to isolate the methanol-rich upper phase from the raffinate lower phase.

In an embodiment, a multi-stage extraction arrangement 108, wherein the extraction unit 102 and separation chamber 104 are used iteratively with fresh methanol for multiple cycles.

In an embodiment, an evaporation unit 110 is configured to heat the combined methanol-rich extracts at approximately 70° C. to evaporate methanol and isolate a crystalline naphthalene residue.

In an embodiment, a purification unit 112 is connected to the evaporation unit comprising a series of washing chambers for sequentially washing the crystalline residue with dilute sulphuric acid, caustic solution, and water.

In an embodiment, a drying and melting chamber 114 is coupled to the purification unit to heat the washed residue to approximately 100° C. to remove moisture and convert the naphthalene into a liquid state.

In an embodiment, a cooling and crystallization unit 116 is in continuation with the drying and melting chamber to allow the liquid naphthalene to cool to approximately 25° C. to obtain solid naphthalene crystals.

In another embodiment, the naphthalene is recovered from rerun column bottom using a cooling and decanting process, comprising: a sample container configured to hold and divide rerun column bottom into separate aliquots; a temperature control unit comprising a refrigeration chamber for maintaining one sample at approximately 16° C. and an ambient temperature chamber for maintaining another at 25° C.; a settling unit configured to allow the 16° C. sample to return to room temperature over 3 to 4 hours for phase separation; a decanting mechanism to remove the supernatant liquid layer from the settled naphthalene; a filtration unit comprising Whatman-1 filter paper and an absorbent tissue support placed below the filter to remove residual solvent from the collected solids; a solvent treatment unit configured to dissolve the filtered crystals in acetone; a heating plate maintained at 100±2° C. to evaporate acetone from the solution; and a crystallization chamber for cooling the sample to room temperature and collecting dried naphthalene crystals.

The system 100 further comprising: a multi-stage water washing unit configured for washing the filtered naphthalene crystals 3-4 times; a drying oven operable at 100±2° C. for moisture removal from the washed crystals; and a crystallization chamber configured to cool and stabilize the dried crystals at room temperature to allow complete formation of solid naphthalene.

In a further embodiment, the crystallization unit is configured to allow solidification within approximately 30 minutes at ambient temperature, wherein the drying oven is configured to operate at approximately 100±2° C. for approximately 2 hours. In an embodiment, the phase separation chamber is configured with a transparent housing and time-controlled settling feature to allow the mixture of methanol and rerun column bottom to remain undisturbed for a period of 5 to 6 hours, thereby facilitating complete gravitational separation of the methanol-rich phase from the denser raffinate phase, wherein the purification unit comprises a series of interconnected washing chambers, each dedicated to a specific washing solution selected from dilute sulphuric acid, caustic solution, and distilled water, wherein the crystalline residue is transferred sequentially through each chamber to achieve step-wise removal of acid-soluble, base-soluble, and water-soluble impurities respectively.

In an embodiment, the phase separation chamber is engineered with a transparent housing and a built-in time-controlled settling mechanism that allows precise visual monitoring and unagitated retention of the solvent-feed mixture for a prolonged period-typically 5 to 6 hours. The transparent housing is constructed from chemically inert materials such as borosilicate glass or clear acrylic polymer, allowing real-time observation of the biphasic stratification that naturally occurs between the lighter methanol-rich extract and the denser, dark raffinate phase originating from the rerun column bottom sample. The chamber is designed with a cylindrical or conical geometry to minimize turbulence during pouring and to promote laminar settling conditions.

A key aspect of this embodiment is the time-controlled settling feature, which may be implemented using a programmable timing circuit or a manual valve system that locks the chamber for a defined period. For instance, once 100 mL of methanol is added to 100 mL of rerun column bottom and thoroughly mixed, the mixture is left in the phase separation chamber without agitation. Over the 5 to 6-hour undisturbed settling period, gravitational differentiation occurs: polar and semi-polar aromatics preferentially partition into the methanol phase, while heavy tar-like fractions and polymeric contaminants settle into the raffinate phase at the bottom.

Upon clear visual formation of the interface between the two phases—facilitated by the transparency of the chamber—the methanol-rich phase is decanted or drained for further purification. This decanted extract, which may still contain soluble impurities, is transferred into the purification unit.

The purification unit comprises a series of interconnected washing chambers, each designated to perform a specific chemical cleanup operation. The first chamber contains dilute sulphuric acid (e.g., 5-10% H₂SO₄), which reacts with and removes basic nitrogenous impurities or unsaturated compounds. The extract is gently agitated and then allowed to settle before decanting into the second chamber containing a mild caustic solution, such as 5% sodium hydroxide, which neutralizes and removes acidic impurities like phenolics or sulfurous compounds. Finally, the solution passes through a distilled water chamber to eliminate any residual acid or base carryover, ensuring a neutral pH prior to crystallization.

Each washing chamber is equipped with overflow channels or controlled valves to enable clean transfer of the extract, and stirring paddles or ultrasonic agitators may be integrated for consistent reagent contact. The crystalline naphthalene residue—precipitated at low temperatures following solvent evaporation—is then subjected to the same washing sequence, ensuring that any occluded or surface-bound chemical residues are effectively removed. This embodiment achieves multi-level purification by combining physical settling with targeted chemical washing, enabling high recovery and purity of naphthalene suitable for industrial reuse or analytical-grade applications.

The extraction unit is designed as a chemically resistant borosilicate or stainless-steel vessel capable of accommodating 100 milliliters of rerun column bottom sample and an equal volume of methanol. It is fitted with a magnetic or mechanical agitator configured to operate at a defined speed sufficient to achieve homogeneous dispersion of the methanol throughout the hydrocarbon matrix for a duration of approximately two minutes. The agitation facilitates intimate phase contact and promotes the dissolution of naphthalene into the methanol solvent phase. The extraction vessel is equipped with a vapor-tight seal to prevent methanol loss during agitation and includes temperature control features to maintain ambient conditions at approximately 25° C., ensuring reproducible solvent partitioning behavior.

The phase separation chamber is fluidly connected to the extraction unit via a transfer conduit and serves as a quiescent environment for gravity-driven segregation of the immiscible methanol and raffinate phases. It is typically constructed from glass or stainless steel and configured to minimize turbulence upon transfer of the mixed liquid. The chamber geometry includes a conical base with a controlled inclination angle to accelerate coalescence of dispersed droplets and facilitate the formation of a clear interfacial boundary. The mixture is allowed to stand undisturbed for a period of five to six hours, during which density and polarity differences between the phases cause complete stratification.

The separation mechanism comprises a siphon or drain assembly connected to the phase separation chamber, enabling selective withdrawal of the methanol-rich upper phase while leaving the raffinate phase undisturbed. The siphon is height-adjustable, permitting precise alignment with the liquid interface to prevent cross-contamination between layers. The outlet of the separation mechanism is linked to a collection flask for recovery of the methanol phase, which contains the extracted naphthalene solute. The design allows automated or manual operation and can be configured for batch or continuous processing.

The multi-stage extraction arrangement utilizes the same extraction and separation assemblies iteratively in successive cycles with fresh methanol solvent. After each extraction, the raffinate phase is reintroduced into the extraction unit with a new aliquot of methanol, ensuring progressive depletion of residual naphthalene. The cyclic configuration may include automated valves and pumps to regulate solvent input, discharge timing, and cycle count, thereby enabling consistent solvent-to-sample ratios and reproducible extraction efficiency across multiple passes.

The evaporation unit is constructed as a rotary evaporator or vacuum distillation apparatus, wherein the combined methanol-rich extracts are subjected to controlled heating at approximately 70° C. The system operates under reduced pressure (typically 200-300 mbar) to lower the boiling point of methanol and enable efficient solvent removal without degrading naphthalene. The evaporator includes a condenser coil connected to a receiver flask for methanol recovery and a temperature-regulated water bath to maintain consistent heating. Evaporation is continued until methanol vapor evolution ceases and a concentrated crystalline residue of crude naphthalene remains on the inner flask wall or vessel base.

The purification unit is directly connected to the evaporation assembly and comprises a sequence of three washing chambers arranged in series. Each chamber is equipped to receive the naphthalene residue and introduce washing reagents under controlled agitation. The first chamber contains a 10% dilute sulphuric acid solution to remove basic and metallic impurities, the second chamber holds a 5% sodium hydroxide solution to neutralize residual acidity and convert sulfonated byproducts into soluble salts, and the third chamber supplies deionized water to eliminate any remaining ionic contaminants. Each chamber is fitted with drainage and transfer conduits to allow smooth progression between washing stages without mechanical disruption of the solid mass.

The drying and melting chamber is positioned downstream of the purification sequence and is designed to heat the washed naphthalene residue to approximately 100° C. The chamber incorporates uniform thermal distribution through resistance heating or oil-jacketed circulation, enabling both moisture removal and controlled liquefaction of the naphthalene. The molten state ensures homogeneity and provides a convenient transition into the crystallization stage. A vapor vent or condenser may be included to capture and recycle any volatile emissions during heating. The cooling and crystallization unit is connected in continuation with the drying and melting chamber and facilitates gradual solidification of the liquefied naphthalene. The unit consists of a temperature-controlled chamber or mold assembly in which the molten material is cooled from 100° C. to approximately 25° C. under ambient or regulated airflow conditions. Cooling is achieved at a controlled rate to promote the orderly formation of pure naphthalene crystals with minimal internal stresses. The crystallization unit may include temperature sensors and passive air circulation to ensure uniform cooling and consistent crystal morphology.

In an embodiment, the drying and melting chamber is configured with a thermostatically controlled heating element set to maintain a uniform temperature of approximately 100° C., such that the washed naphthalene residue is simultaneously dehydrated and melted into a homogenous liquid phase for subsequent crystallization. In an embodiment, the drying and melting chamber is specifically engineered to serve a dual function: dehydration and melting of the washed naphthalene residue. The chamber is constructed from thermally conductive and chemically inert materials such as stainless steel or ceramic-coated aluminum to ensure uniform heat distribution and avoid contamination during thermal processing. A thermostatically controlled heating element—such as a nichrome wire heater or a flat-plate resistive heater—encircles or underlies the chamber, and is regulated by a precision temperature control system, typically involving a PID (Proportional-Integral-Derivative) controller connected to a thermocouple embedded in the heating plate. This setup allows for continuous maintenance of the target temperature at approximately 100° C., with minimal deviation (±1° C.), which is critical for preserving the molecular stability of naphthalene while ensuring complete phase transition.

Upon introduction of the washed crystalline naphthalene residue—typically still retaining trace moisture from the final water rinse—the chamber initiates a controlled heating cycle. As the temperature rises steadily to around 100° C., the residual water first evaporates. This is facilitated by the open or semi-open design of the chamber that permits vapor escape without significant heat loss. For example, if 25 grams of wet naphthalene crystals are placed in the chamber, the initial dehydration occurs over 5 to 7 minutes, during which steam condensation can be observed on the interior lid or vent outlet. Once moisture is fully driven off, continued heating causes the solid naphthalene to melt uniformly into a clear, viscous liquid. The homogeneity of the liquid is visually observable and can be further verified by stirring mechanisms or optical sensors detecting transparency and viscosity levels.

This integrated process avoids the need for separate drying ovens and melting baths, thus reducing handling steps and minimizing contamination risks. The resulting molten naphthalene, now devoid of water or residual chemical impurities, is suitable for direct transfer into a crystallization chamber or casting mold. The consistency of the melt ensures that subsequent solidification produces high-purity, structurally uniform naphthalene crystals with reduced risk of occlusions or microvoids. This chamber design is particularly advantageous in continuous or semi-continuous production environments, where precise thermal control directly correlates with product quality and yield.

In an embodiment, the settling unit comprises a passive temperature equalization chamber that enables the previously cooled 16° C. sample to gradually attain room temperature over a span of 3 to 4 hours, wherein this gradual thermal transition enhances the clarity of phase separation between solidified naphthalene and liquid contaminants. In an embodiment, the settling unit is equipped with a passive temperature equalization chamber specifically designed to facilitate a slow and controlled thermal transition of a previously cooled sample, typically at 16° C., to ambient room temperature over a span of 3 to 4 hours. This chamber plays a crucial role in refining the clarity and definition of phase boundaries between solidified naphthalene crystals and coexisting liquid contaminants that may still be present after initial chilling. The chamber itself is constructed using thermally insulating materials such as expanded polystyrene or polyurethane foam-lined stainless steel, creating a microenvironment where external temperature changes are buffered and internal heat exchange occurs in a moderated fashion.

The sample, previously subjected to low temperatures to promote initial naphthalene crystallization, is placed within this chamber in a sealed but thermally conductive container-commonly borosilicate glass or stainless steel. The passive warming process is achieved without the use of active heating elements. Instead, the chamber allows ambient thermal energy to permeate slowly through the insulating barrier, raising the sample temperature at a steady rate of approximately 2-3° C. per hour. This rate of warming is intentional and critical: if temperature increases too rapidly, it may partially remelt the naphthalene or disturb the fragile crystal-liquid interface, leading to turbidity, occlusions, or premature mixing of phases.

For example, a 200 mL methanol extract containing dissolved and partially precipitated naphthalene, cooled to 16° C. in a prior step, is transferred into the chamber and allowed to equilibrate. Over a 4-hour interval, the temperature gently rises to 25-27° C. During this time, residual methanol and non-crystalline impurities in the liquid phase separate more cleanly from the crystallized naphthalene mass. As a result, the solid-liquid interface becomes visually sharper, and the sedimented naphthalene appears more translucent and defined. This clarity is particularly beneficial prior to filtration or decantation, as it enables selective recovery of the solid phase with minimal entrainment of liquid contaminants. The embodiment thus ensures reproducibility and enhances the efficiency of downstream purification, especially in batch processing scenarios where visual cues and interface stability are used as criteria for phase separation readiness.

In an embodiment, the filtration unit includes a disposable filtration assembly having a Whatman-1 grade filter paper positioned over an absorbent tissue substrate within a funnel structure, wherein the tissue absorbs residual filtrate and improves the efficiency of solid crystal recovery with minimal solvent retention.

In an embodiment, the filtration unit is designed as a disposable, gravity-assisted assembly that ensures efficient separation of solid naphthalene crystals from the remaining liquid phase, with special attention to minimizing solvent retention and maximizing recovery of purified crystals. The assembly consists of a standard conical funnel structure—made from inert, solvent-resistant material such as borosilicate glass or PTFE—within which a Whatman-1 grade filter paper is positioned. This filter paper is known for its medium retention (11 μm) and flow rate, making it suitable for capturing moderately sized crystalline solids while allowing solvent and finer impurities to pass through efficiently.

Beneath the filter paper, an absorbent tissue substrate—such as laboratory-grade cellulose wadding or multiple folds of lint-free blotting paper—is strategically placed at the base of the funnel. This absorbent layer serves a dual function: first, it cushions the filter paper and provides added mechanical support under the weight of the collected crystals; second, and more critically, it actively draws and retains residual filtrate that may otherwise pool beneath or around the crystals. This capillary action reduces the tendency of liquid reabsorption by the naphthalene crystals, which are prone to partial dissolution or stickiness if left in prolonged contact with the solvent.

For example, after the passive temperature equalization and crystallization steps, the mixture containing naphthalene crystals and methanol or residual wash water is poured into the funnel. The Whatman-1 paper traps the crystals while the solvent passes through into the collection flask. As the last few milliliters of filtrate move through, the tissue layer absorbs this residual liquid, effectively drying the underside of the filter medium. This reduces drying time downstream and ensures that the crystals collected are not contaminated by retained solvent. Moreover, because the filtration assembly is disposable, cross-contamination between batches is eliminated, making the process particularly suited for small-scale or laboratory environments where purity is paramount. This embodiment significantly enhances the practicality and yield of solid recovery without the need for vacuum filtration or centrifugation, while still achieving high separation efficiency and solvent removal.

In an embodiment, the multi-stage water washing unit comprises three to four sequential rinsing chambers through which the filtered naphthalene crystals are passed, wherein each chamber is flushed with distilled water to ensure complete removal of any adhered chemical agents or residual solvents prior to final drying.

In an embodiment, the multi-stage water washing unit is constructed as a series of three to four sequential rinsing chambers, each engineered to perform a specific decontamination function by progressively removing trace impurities from the filtered naphthalene crystals. These chambers are arranged in a cascade configuration, allowing the crystals to be transferred from one chamber to the next in a linear or rotational path, either manually using inert scoops or via automated transfer mechanisms such as gravity chutes or rotating trays. Each chamber is fabricated from chemically inert and non-leaching materials like PTFE-lined stainless steel or high-grade borosilicate glass to prevent contamination during washing.

Upon completion of filtration, where crude naphthalene crystals are collected, the crystals are introduced into the first rinsing chamber containing distilled water at room temperature. Here, the primary function is to dissolve and dislodge any superficial residues of prior washing agents, such as sulfuric acid or sodium hydroxide, which may remain adsorbed on the crystal surfaces. The crystals are gently agitated for 2 to 3 minutes, either manually or with a mechanical shaker, ensuring uniform exposure to the wash medium.

The partially cleaned crystals are then transferred to the second and third rinsing chambers, where fresh distilled water is flushed in counterflow relative to crystal movement—i.e., the cleanest water contacts the cleanest crystals at the final stage. This technique minimizes recontamination and enhances rinsing efficiency. Each chamber may be equipped with gentle stirring paddles or ultrasonic agitators to promote detachment of ionic or polar residues without damaging the crystalline structure.

For instance, in a laboratory-scale setup handling 50 grams of naphthalene crystals, each chamber is filled with approximately 200 mL of distilled water, and the crystals are immersed for 2-5 minutes per chamber. Conductivity or pH sensors installed at the chamber outlets can monitor the washing progress, ensuring that by the time crystals exit the final rinsing stage, the rinse water approaches neutral pH and near-zero conductivity-indicating effective removal of all water-soluble impurities and residual solvents.

This multi-stage design ensures a high degree of purity by sequential dilution and removal of surface-bound contaminants. The step-wise approach also prevents localized reabsorption of impurities and prepares the crystals for final drying, free from chemical traces that could affect melting behavior, optical clarity, or downstream analytical performance.

In an embodiment, the crystallization chamber comprises a thermally insulated container configured to receive the molten naphthalene and maintain it at ambient temperature conditions for approximately 30 minutes, thereby facilitating the uniform and controlled formation of solid naphthalene crystals with improved purity and stability.

In an embodiment, the crystallization chamber is designed as a thermally insulated container that enables controlled solidification of molten naphthalene under stable ambient temperature conditions, ensuring uniform crystal growth and improved material purity. The chamber is typically constructed from double-walled stainless steel or glass with an internal vacuum or foam insulation layer that minimizes heat loss through conduction and convection. This design ensures that once the molten naphthalene-heated to approximately 100° C. in the preceding melting chamber—is transferred into the crystallization vessel, the rate of cooling is slow and predictable, rather than abrupt, which is essential for optimal crystal morphology.

Once the molten naphthalene is poured into the chamber, which may include a shallow tray or mold made of chemically inert materials such as PTFE or borosilicate glass, it is allowed to stand undisturbed for approximately 30 minutes. The ambient temperature, typically in the range of 25-28° C., facilitates gradual thermal dissipation from the molten mass. The thermal insulation delays rapid surface cooling that could otherwise result in uneven nucleation or amorphous solid formation. Instead, the molten naphthalene cools from the periphery inward, promoting consistent crystal growth throughout the bulk volume. To further enhance uniformity, the chamber may optionally include a lid with small pressure-equalizing vents that allow solvent vapor to escape without disrupting the thermal environment.

For example, a 100 mL batch of molten naphthalene poured into a flat crystallization mold within the insulated chamber will begin to exhibit surface solidification within 5-7 minutes, while the full mass solidifies evenly over the remaining period. The resulting crystals are typically well-defined, with a translucent or pearlescent appearance, indicative of high purity and minimal inclusion of trapped gases or solvents.

By preventing temperature gradients and environmental fluctuations during the solidification phase, this embodiment ensures that the final naphthalene crystals are physically stable, less prone to sublimation during storage, and chemically free from occluded impurities. The slow, unforced crystallization also results in larger, more easily filterable crystals, which improve the overall yield and handling in subsequent packaging or analytical procedures. Thus, the insulated crystallization chamber plays a critical role in the final stage of the purification process, bridging thermal processing and material recovery with precision and reproducibility.

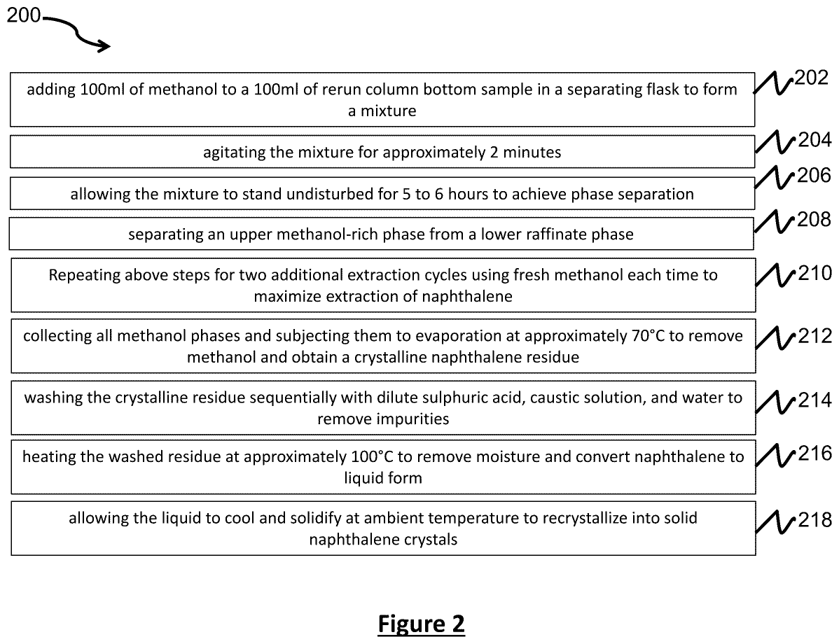

FIG. 2 illustrates a flow chart of a method for recovering naphthalene from a rerun column bottom sample in accordance with an embodiment of the present disclosure. At step 202, the method 200 includes adding 100 ml of methanol to a 100 ml of rerun column bottom sample in a separating flask to form a mixture.

At step 204, the method 200 includes agitating the mixture for approximately 2 minutes.

At step 206, the method 200 includes allowing the mixture to stand undisturbed for 5 to 6 hours to achieve phase separation.

At step 208, the method 200 includes separating an upper methanol-rich phase from a lower raffinate phase.

At step 210, the method 200 includes repeating above steps for two additional extraction cycles using fresh methanol each time to maximize extraction of naphthalene.

At step 212, the method 200 includes collecting all methanol phases and subjecting them to evaporation at approximately 70° C. to remove methanol and obtain a crystalline naphthalene residue.

At step 214, the method 200 includes washing the crystalline residue sequentially with dilute sulphuric acid, caustic solution, and water to remove impurities.

At step 216, the method 200 includes heating the washed residue at approximately 100° C. to remove moisture and convert naphthalene to liquid form.

At step 218, the method 200 includes allowing the liquid to cool and solidify at ambient temperature to recrystallize into solid naphthalene crystals.

In another embodiment, the separation time of 5 to 6 hours is optimized to achieve maximum separation between methanol and raffinate phases.

In a further embodiment, the ambient temperature is preferably 25° C.

In one of the above embodiments, the naphthalene is recovered from rerun column bottom using a cooling and decanting process, comprising dividing a rerun column bottom into two sample sets. Then, placing a first sample at ambient room temperature preferably ˜25° C. and a second sample at a lower temperature preferably ˜16° C. then, observing that the second sample at 16° C. undergoes partial solidification of naphthalene. Then, allowing the second sample to return to room temperature over a period of 3 to 4 hours to promote separation and settling of solid naphthalene. Then, decanting the upper liquid layer and filtering the remaining slurry using Whatman-1 filter paper. Then, placing a tissue paper beneath the filter paper to remove residual liquid from the solid crystals. Then, dissolving the collected naphthalene crystals in acetone. Then, evaporating the acetone by placing the solution on a hot plate at approximately 100±2° C. until dry. Thereafter, cooling the resulting material to room temperature to obtain dried naphthalene crystals.

In one embodiment, solidification of naphthalene is initiated by maintaining the sample at approximately 16° C. for a predefined duration, wherein acetone is used as a solvent to redissolve and recrystallize the filtered naphthalene for improved purity.

The method 200 further comprising: washing the recovered solid crystals with water 3-4 times to remove impurities. Then, placing the crystals in an oven operated at 100±2° C. for 2 hours to remove moisture. Thereafter, removing the crystals from the oven and allowing them to rest at room temperature to settle for approximately 30 minutes. In an embodiment, the agitation of the mixture in step 204 is carried out by using a magnetic stirrer, wherein such agitation ensures thorough dispersion of the methanol within the rerun column bottom to maximize contact area for efficient extraction of naphthalene, and wherein each subsequent extraction cycle performed in step 210 involves the addition of 100 ml of fresh methanol to the same volume of previously extracted raffinate, followed by agitation and phase separation, wherein each iteration is configured to progressively increase the yield of extracted naphthalene into the methanol-rich phase.

In an embodiment, the agitation of the mixture in step 204 is executed using a magnetic stirrer, which provides uniform and controlled mixing of methanol with the rerun column bottom sample. This setup typically involves placing a magnetic stir bar within the separating flask and situating the flask on a motorized magnetic stirring platform capable of adjustable rotational speeds. The magnetic stirrer ensures that methanol is evenly dispersed throughout the viscous and heterogeneous matrix of the rerun column bottom, thereby maximizing the interfacial contact area between the solvent and solute phases. This enhanced contact is critical for the effective solubilization and extraction of naphthalene, which preferentially partitions into the polar methanol phase due to its solubility characteristics under the applied conditions.

During agitation, typically lasting for approximately two minutes, the methanol penetrates into the viscous matrix of the rerun column bottom, facilitating the mobilization of semi-volatile aromatic hydrocarbons such as naphthalene from the dense tar-like phase into the methanol-rich extract. The resulting emulsion is temporarily stable, allowing for deeper molecular interaction before gravitational settling is initiated. Once agitation ceases, the mixture is allowed to rest in a phase separation chamber for 5 to 6 hours, during which two distinct layers form: a lighter, methanol-rich upper phase containing dissolved naphthalene, and a denser raffinate phase with unextractable heavy residues.

To maximize recovery, step 210 involves a series of subsequent extraction cycles using fresh aliquots of methanol. In each iteration, 100 ml of fresh methanol is added to the same volume of previously extracted raffinate. The procedure is repeated: magnetic stirring for uniform dispersion, followed by static settling in the transparent phase separation chamber. Each cycle extracts residual naphthalene that was not fully removed in the prior iteration, effectively improving total yield without requiring high-pressure or thermal-assisted extraction.

For instance, in the first extraction cycle, the methanol phase may recover 60-70% of the available naphthalene. A second extraction with fresh methanol on the same raffinate may recover an additional 15-20%, and a third extraction another 5-10%, depending on process efficiency and feedstock composition. The serial use of fresh methanol ensures that the concentration gradient remains favorable for continued extraction across cycles. By incorporating a magnetic stirrer for consistent agitation and performing successive extraction cycles with fresh solvent, this embodiment ensures a scalable and efficient recovery process, significantly enhancing the purity and overall yield of naphthalene from complex industrial by-products such as rerun column bottoms.

In an embodiment, the evaporation step 212 involves transferring the combined methanol-rich extracts into an evaporation container and applying a consistent heat of approximately 70° C., wherein evaporation is continued until all volatile methanol content is visibly removed, resulting in the formation of solid naphthalene crystals at the base of the container, wherein the sequential washing of the crystalline residue in step 214 is performed by first treating the residue with dilute sulphuric acid to remove acid-soluble impurities, then with a caustic solution to neutralize and remove any remaining acidic or tarry substances, and finally with distilled water to eliminate residual chemical traces, thereby improving the purity of the recovered naphthalene.

In an embodiment, the evaporation step 212 is designed to recover solid naphthalene crystals from the methanol-rich extract through controlled thermal evaporation of the solvent. The combined methanol extracts obtained from one or more extraction cycles are transferred into a flat-bottom evaporation container made of heat-resistant, chemically inert material such as borosilicate glass. The container is placed on a thermostatically controlled hotplate or within a convection drying oven that maintains a stable temperature of approximately 70° C. carefully chosen to remain below methanol's boiling point (64.7° C.) to ensure complete vaporization while avoiding rapid boiling that might cause splashing or loss of valuable product.

As heat is applied, the methanol gradually evaporates in a uniform manner, and no stirring is required during this phase to avoid disturbing the forming crystal lattice. Over the course of the evaporation—typically 1 to 2 hours depending on volume—the solution's clarity diminishes, and visible white to off-white crystalline deposits of naphthalene begin to appear and settle at the base of the container. The low volatility of naphthalene under these conditions ensures it remains as a solid residue, while the methanol vapor escapes. To ensure worker safety and environmental compliance, the setup may be enclosed under a fume hood or equipped with a simple vapor condensation system.

Once all the volatile methanol content is visibly removed—indicated by the complete dryness of the crystal bed and absence of solvent sheen—the container is cooled to room temperature, and the dried crystalline residue is collected for the washing phase. The sequential washing of the crystalline residue in step 214 is performed to eliminate trace chemical impurities that co-precipitate or adhere during evaporation.

Initially, the residue is treated with dilute sulphuric acid (5-10%) to dissolve and remove basic impurities, organo-nitrogen compounds, or certain unsaturated hydrocarbons that are acid-soluble. The acid wash is typically carried out by gentle immersion and stirring for 2-3 minutes, after which the acid is decanted. Next, a mild caustic solution (5% NaOH) is applied to neutralize any remaining acidic substances and remove tarry residues or sulfur-containing compounds that are base-soluble. The caustic wash also contributes to deodorization and stabilization of the crystals. Finally, the residue is subjected to at least two rounds of distilled water rinsing to flush away all remaining traces of acid and base, and to restore the crystalline material to a neutral pH condition.

For example, a batch of 10 grams of crude naphthalene crystals subjected to this triple-washing sequence typically results in a final product with over 95% purity by weight, with significantly reduced levels of color, odor, and chemical reactivity. The controlled evaporation and meticulously sequenced washing steps thus enable the recovery of highly pure naphthalene suitable for further chemical synthesis, laboratory analysis, or reuse in closed industrial cycles.

In an embodiment, the step 216 of heating the washed residue is carried out by placing the crystals in a preheated oven maintained at approximately 100° C. for a sufficient duration to ensure complete removal of surface and entrapped moisture, and to uniformly melt the naphthalene without degradation of its chemical composition, and wherein the recrystallization step 218 is performed by transferring the molten naphthalene into a clean, inert container and allowing it to cool undisturbed at ambient temperature of approximately 25° C., whereby gradual cooling promotes the formation of uniform, high-purity solid naphthalene crystals through controlled crystallization dynamics.

In an embodiment, step 216 of the process involves subjecting the washed naphthalene residue to a precisely controlled heating operation aimed at both dehydrating and melting the solid material without compromising its chemical integrity. The crystals, having undergone sequential acid, base, and water washes to remove a wide spectrum of soluble impurities, are gently transferred to a preheated oven set at a uniform temperature of approximately 100° C. This temperature is carefully chosen based on the melting point of naphthalene (around 80-82° C.), allowing for complete phase transition into a liquid while also facilitating the evaporation of any residual surface or interstitial moisture that could have been retained during the washing and rinsing steps. The oven, typically a laboratory-grade convection unit with accurate thermal regulation, ensures that the heating is evenly distributed across the sample without inducing localized overheating, which could otherwise cause sublimation, discoloration, or thermal decomposition of the naphthalene.

This drying and melting operation is maintained for a duration sufficient to ensure the entire mass achieves a uniform, bubble-free molten state—generally between 15 to 25 minutes depending on batch volume. During this phase, care is taken to avoid agitation, which can introduce microbubbles or impurities into the liquid mass. Once complete melting and dehydration are confirmed—either visually or via weight stability checks—the molten naphthalene is carefully decanted or poured into a clean and inert crystallization container in step 218.

The recrystallization step is then initiated by allowing the molten naphthalene to cool under ambient temperature conditions, typically around 25° C., in an undisturbed environment. The container is made of an inert, non-reactive material such as PTFE, borosilicate glass, or stainless steel to prevent contamination and facilitate easy removal of the solidified product. The ambient cooling process is allowed to proceed naturally, without external fans or forced convection, ensuring that the temperature gradient from the container walls to the center of the molten mass is gradual and uniform.

This gradual thermal descent encourages controlled nucleation and ordered crystal growth, yielding large, uniform naphthalene crystals with a defined monoclinic structure and minimal occlusions. Unlike rapid quenching, which may produce amorphous solids or fragmented crystals, the slow cooling approach taken in this embodiment enhances purity, stability, and overall quality of the recovered solid naphthalene. For instance, crystallization over 30 to 45 minutes results in well-formed, translucent white crystals with minimal discoloration or residual odor, which are then suitable for downstream use in chemical synthesis, analytical applications, or as a high-purity feedstock for other industrial processes. This embodiment thus ensures that the final product meets stringent quality standards by employing precise thermal processing and passive crystallization control to eliminate moisture, preserve chemical fidelity, and achieve high-purity solid-state recovery.

In an embodiment, the cooling of the rerun column bottom to approximately 16° C. is achieved by placing the sample in a temperature-controlled chamber, wherein solidification of naphthalene is initiated and upon gradual warming to ambient room temperature over 3 to 4 hours, separation of solid naphthalene from the supernatant liquid is achieved due to differential solubility and phase density, and wherein the filtration of the cooled and settled slurry is performed by pouring the material onto Whatman-1 filter paper supported by an underlying tissue paper placed inside a funnel, such that the tissue paper absorbs residual liquid and aids in the efficient drying and isolation of solid naphthalene particles.

In an embodiment, the selective solidification and recovery of naphthalene from the rerun column bottom is achieved through a carefully controlled temperature manipulation strategy that exploits the physical properties of naphthalene, specifically its crystallization behavior at lower temperatures. The initial step involves cooling the methanol-extracted sample to approximately 16° C., which is carried out by placing the liquid extract in a temperature-controlled chamber, such as a refrigerated environmental chamber or laboratory cooling unit equipped with feedback control. This unit is calibrated to hold the internal atmosphere steadily at 16° C., a temperature sufficiently low to initiate the nucleation of naphthalene crystals without freezing other components or solvents that may be present.

As the sample cools within this environment, naphthalene—due to its relatively low solubility in cold methanol—begins to separate out of the solution in crystalline form. The solidification proceeds gradually over a period of one to two hours, forming discernible white or translucent needle-like or flake-like crystalline structures at the bottom of the container. At this point, the sample, now a slurry containing a solid-liquid mixture, is removed from the cooling chamber and allowed to passively warm up to ambient room temperature (approximately 25° C.) over a span of 3 to 4 hours. This gradual warming is essential to promote clear separation between the solid naphthalene and the remaining supernatant liquid, which may contain other soluble impurities, tars, or uncrystallized aromatic compounds.

The differential solubility and phase density of naphthalene in relation to the surrounding matrix at these transitional temperatures enable the crystalline phase to remain intact and settle at the bottom, while lighter or more volatile contaminants remain suspended in the liquid phase. Once this stratification has occurred, the slurry is ready for filtration.

Filtration is performed by carefully decanting or pouring the settled mixture into a standard funnel apparatus lined with Whatman-1 grade filter paper, which is known for its medium flow rate and particle retention capability (˜11 μm). Beneath the filter paper, an absorbent tissue paper is placed to act as a secondary substrate. This tissue paper serves two critical functions: first, it absorbs any residual methanol or moisture that may pass through the filter paper, accelerating the drying of the solid naphthalene; second, it prevents pooling at the funnel tip, thereby promoting a more efficient and uninterrupted flow of the filtrate.

This layered filtration design ensures that the solid naphthalene crystals remain on the filter surface, free from contamination by reabsorbed liquid, while the solvent is drawn away by capillary action into the tissue. The result is a more complete and rapid separation of solid product from liquid waste. After filtration, the collected solid may either be used directly or subjected to additional washing and drying steps, depending on the required purity level.

In an embodiment, the filtered solid naphthalene crystals are redissolved in acetone to purify the material, and the solution is placed on a hot plate maintained at a temperature of 100±2° C., wherein acetone is evaporated until complete dryness is observed and purified naphthalene is recovered in solid form, and wherein after washing the solid naphthalene crystals 3 to 4 times with water to remove soluble impurities, the crystals are transferred to an oven operated at a stable temperature of 100±2° C. for a continuous drying cycle of approximately 2 hours, followed by cooling at room temperature for a duration of about 30 minutes to stabilize the crystalline structure and enhance solid recovery.

In an embodiment, the filtered solid naphthalene crystals, which may still contain residual organic impurities or color bodies even after initial methanol extraction and water-based washing, undergo a secondary purification step using acetone as a selective solvent. This step capitalizes on the high solubility of naphthalene in acetone at elevated temperatures, combined with the ability of acetone to exclude many non-aromatic or polymeric impurities.

The solid crystals are added to a clean glass beaker containing a sufficient volume of analytical-grade acetone, typically in a ratio of about 1 gram of crystal per 10-15 ml of solvent. Gentle stirring or swirling may be employed to assist complete dissolution of the crystals, forming a clear or lightly tinted solution.

The solution is then placed on a thermostatically controlled hot plate maintained at approximately 100±2° C. This elevated temperature accelerates the evaporation of acetone, which has a boiling point of around 56° C., ensuring rapid removal of the solvent without inducing sublimation or thermal decomposition of naphthalene. As the acetone evaporates, naphthalene begins to recrystallize directly within the container, forming a fine crystalline or flaky solid layer. Complete dryness is typically achieved within 10 to 20 minutes depending on solvent volume and ambient humidity, and the purified naphthalene is visibly distinguishable as a snow-white to pale crystalline mass at the base of the beaker.

Following this evaporation-driven reprecipitation step, the recrystallized naphthalene is subjected to an aqueous rinsing procedure. The solid is washed three to four times with distilled water to remove any acetone-soluble byproducts, acid- or base-derived residues, and other water-soluble contaminants that may have been co-precipitated or adsorbed during previous processing stages. Each rinse involves gently pouring or swirling distilled water over the crystals, allowing a brief contact period, and then decanting the supernatant. These wash cycles help improve not only the chemical purity but also the odor profile and physical consistency of the product.

To ensure complete dehydration of the washed crystals, they are next transferred to a drying oven set to a stable temperature of 100±2° C. The drying chamber is typically a laboratory-grade convection or vacuum oven designed to uniformly remove surface and interstitial water without causing thermal degradation. The drying cycle is maintained continuously for approximately 2 hours, which has been empirically found to be sufficient to reduce moisture content below 0.1% by weight. The long, low-temperature drying cycle also ensures that the final product does not retain any residual solvents that could interfere with downstream usage.

After this drying step, the solid naphthalene is removed from the oven and allowed to cool at ambient room temperature (approximately 25° C.) for a duration of about 30 minutes. This passive cooling period is critical for stabilizing the crystalline structure, allowing recrystallized domains to reorient and consolidate under thermodynamic equilibrium. The resulting product is a chemically pure, physically stable form of solid naphthalene, characterized by its uniform crystal morphology, improved hardness, and high recovery yield—suitable for reuse in analytical chemistry, dye intermediates manufacturing, or other high-purity industrial applications.

In an embodiment, the step of methanol evaporation is executed by introducing the combined methanol phases into a rotary evaporator having a glass evaporation flask partially immersed in a thermostatically controlled water bath set to 70° C., wherein the bath temperature is continuously regulated by a feedback-controlled thermistor circuit, wherein the evaporator is operated under a vacuum pressure of 200-250 mbar generated by a diaphragm vacuum pump connected through a cold trap containing dry ice to condense residual methanol vapors, wherein the rotation speed of the flask is fixed at 90 rpm using a servo drive motor, wherein the condensate outlet is connected to a borosilicate receiver flask with a three-way stopcock for collection and vent balancing, and wherein the evaporation continues until the internal temperature of the flask stabilizes at 70° C. for a minimum period of 20 minutes to ensure complete solvent removal under steady thermal conditions.

In one preferred configuration, the step of methanol evaporation is carried out through a precisely controlled rotary evaporation assembly designed to achieve complete solvent removal while preserving the structural and chemical integrity of the naphthalene fraction. The combined methanol extracts obtained from successive extraction cycles are transferred into a borosilicate glass evaporation flask that is partially immersed in a thermostatically regulated water bath maintained at approximately 70° C. The partial immersion ensures uniform heat distribution across the liquid surface and minimizes splashing or bumping effects that often occur under reduced pressure. The water bath temperature is continuously monitored and adjusted through a feedback-controlled thermistor circuit that maintains thermal equilibrium within narrow limits, thus providing stable vaporization conditions without allowing the internal flask temperature to exceed the threshold that could induce premature crystallization or thermal decomposition of naphthalene.

A diaphragm vacuum pump connected to the vapor outlet maintains an operating vacuum between 200 and 250 mbar, which effectively lowers the boiling point of methanol to around 35-40° C., ensuring that the evaporation proceeds at moderate thermal energy levels. The vacuum line is routed through a cold trap containing dry ice pellets, serving to condense escaping methanol vapors and prevent backstreaming of condensate into the evaporation flask. The rotation of the flask at a fixed speed of 90 revolutions per minute by a servo-controlled drive motor generates a thin, continuously renewed liquid film on the inner surface of the flask, thereby enhancing the mass transfer rate and accelerating solvent removal. The gentle rotation further prevents local overheating and allows for even exposure of the solution to the heating surface.

The methanol vapors generated during the process pass through the condenser coil, where they are liquefied and directed into a borosilicate receiver flask fitted with a three-way stopcock. This arrangement allows for alternate collection and venting, thereby maintaining a consistent internal pressure throughout the evaporation run. The evaporation step is continued until the temperature inside the rotating flask stabilizes at approximately 70° C. for at least twenty minutes, which serves as an indicator that nearly all volatile components have been removed and only the viscous, concentrated naphthalene-rich residue remains. The process parameters-temperature, rotation speed, and pressure—are optimized to achieve a balanced thermal profile, enabling high recovery efficiency of methanol while preserving the purity and crystalline morphology of naphthalene. The synergistic interaction between controlled vacuum, dynamic rotation, and precise thermal regulation ensures a reproducible solvent removal process that minimizes energy consumption and eliminates residual solvent traces that could interfere with subsequent washing or recrystallization stages.

In an embodiment, agitation of the mixture is performed by a magnetic stirrer assembly consisting of a PTFE-coated stir bar having a length of 40 mm and a diameter of 8 mm, positioned centrally at the base of a conical separating flask with a 60° cone angle, wherein agitation is maintained at a speed of 600 rpm for 120 seconds under a constant ambient temperature of 25±1° C., wherein the stirring is followed by immediate quiescence of the mixture in the same vessel without external vibration, and wherein the vessel is fitted with a vapor-tight Teflon stopper to prevent methanol loss during agitation, the stirring cycle being repeated identically for each of the two subsequent extraction iterations with fresh methanol, using identical volumetric ratios and vessel geometry to maintain reproducibility across extraction stages. In this configuration, agitation of the sample-methanol mixture is achieved through a precision-controlled magnetic stirring system that ensures uniform phase contact and reproducible extraction kinetics across all experimental runs. The assembly employs a polytetrafluoroethylene (PTFE)-coated magnetic stir bar with a length of 40 millimeters and a diameter of 8 millimeters, positioned concentrically at the base of a conical separating flask designed with a 60-degree cone angle. The geometry of the flask promotes efficient vortex formation during agitation and facilitates rapid phase disengagement once stirring ceases. The PTFE coating of the stir bar provides chemical inertness against methanol and hydrocarbon components present in the rerun column bottom, preventing any catalytic degradation or introduction of particulates during mixing.

The agitation is carried out at a constant rotational speed of approximately 600 revolutions per minute, calibrated through a closed-loop control of the magnetic drive, and sustained for precisely 120 seconds. This duration and speed combination have been optimized experimentally to maximize solvent-solute interaction without inducing stable emulsions that could impede subsequent phase separation. The operation is conducted under an ambient temperature of 25±1° C., ensuring consistent solvent viscosity and diffusivity parameters across batches, thereby contributing to reproducible mass transfer rates. The flask is sealed with a vapor-tight Teflon stopper that prevents methanol volatilization during agitation, maintaining the solvent-to-feed ratio constant throughout the process and preserving extraction efficiency.

Immediately after the stirring cycle concludes, the mixture is allowed to stand undisturbed in the same vessel, ensuring complete quiescence of the system and facilitating spontaneous gravitational separation of the methanol-rich and hydrocarbon-rich layers. The absence of external vibration or mechanical perturbation during this settling phase ensures that the interface remains well-defined, enabling precise phase decantation in subsequent steps. The identical stirring and settling protocol is repeated for two additional extraction cycles, each employing freshly measured methanol volumes and identical vessel geometry, to maintain experimental consistency and maximize solute recovery. The replication of mechanical and thermal conditions across successive extractions ensures cumulative diffusion-driven recovery of naphthalene molecules while preventing solvent saturation effects, thus enhancing the overall yield through synergistic phase equilibria. This precisely controlled agitation sequence not only ensures high reproducibility but also provides a balance between efficient solute desorption and minimal energy input. The synergistic action of optimized stir speed, controlled temperature, and identical vessel geometry produces a fine equilibrium between extraction efficiency and process stability, resulting in a cleaner phase interface and improved downstream evaporation performance.

In an embodiment, the sample cooling to 16° C. is performed by placing the rerun column bottom in a double-jacketed glass container connected to a recirculating chiller using ethanol as a coolant fluid, wherein the coolant inlet and outlet temperatures are controlled within ±0.5° C. by an integrated proportional-integral-derivative (PID) temperature controller, wherein the cooling rate is maintained at 1.5° C. per minute by adjusting the coolant flow rate through a needle valve, wherein the temperature of the rerun column sample is monitored using a calibrated platinum resistance thermometer inserted into the liquid phase at mid-depth, wherein after reaching 16° C., the system is held isothermally for 4 hours without agitation, and wherein the subsequent warming to room temperature is achieved naturally over 3 hours without forced heating to allow the formation of visible naphthalene solids prior to decantation. In this embodiment, the controlled cooling of the rerun column bottom sample to a precise temperature of 16° C. is carried out through a double-jacketed borosilicate glass vessel engineered to provide uniform heat exchange and eliminate local thermal gradients that could otherwise lead to uneven nucleation of naphthalene. The jacketed vessel is interfaced with a closed-loop recirculating chiller, which circulates ethanol as the cooling fluid owing to its high thermal conductivity and stable viscosity at sub-ambient temperatures. The inlet and outlet ports of the jacket are connected via insulated tubing to minimize external heat gain, ensuring that the cooling bath remains thermally isolated from environmental fluctuations. The ethanol coolant temperature is continuously monitored and regulated within a tolerance of ±0.5° C. using an integrated proportional-integral-derivative (PID) temperature controller, which dynamically adjusts compressor power and coolant flow to maintain a steady thermal gradient.

The cooling rate is carefully modulated to 1.5° C. per minute by fine-tuning the coolant flow through a precision needle valve installed at the chiller outlet. This slow, linear cooling trajectory prevents thermal shock and allows controlled supersaturation of dissolved naphthalene in the heavy hydrocarbon matrix, thereby promoting the gradual onset of crystallization rather than abrupt solid precipitation. The internal temperature of the rerun column bottom sample is continuously tracked using a calibrated platinum resistance thermometer (Pt100 type) inserted into the liquid phase at mid-depth to ensure an accurate reflection of bulk temperature rather than surface or wall conditions. Once the target temperature of 16° C. is achieved, the system is maintained in a strictly isothermal state for a period of four hours without any mechanical agitation. This quiescent holding period allows the molecular reorganization and crystal lattice formation of naphthalene to progress unhindered, enabling the development of well-defined crystalline domains and facilitating gravity-assisted solid-liquid segregation.

After the isothermal hold, the sample is allowed to gradually warm to room temperature, approximately 25° C., over a duration of three hours under natural convective conditions without the application of external heating. This gradual temperature relaxation provides a favorable environment for the growth of visible naphthalene solids while simultaneously reducing the solubility of naphthalene in the surrounding liquid phase. The thermal gradient between the cooled interior and ambient surroundings enhances the sedimentation of the crystalline phase, thereby simplifying subsequent decantation and minimizing entrainment of impurities. This precisely controlled cooling-warming sequence creates a synergistic effect between the rate of supersaturation and the thermodynamic stability of crystal nuclei, resulting in the formation of pure, well-structured naphthalene crystals with minimal inclusion of residual hydrocarbons. The slow cooling and natural warming together ensure that the process remains energy-efficient and self-regulated while maintaining the chemical integrity of the recovered compound. The combination of PID-regulated thermal control, controlled cooling rate, and isothermal holding yields reproducible crystallization behavior and consistent purity levels, forming a critical step that bridges solvent extraction and solid recovery with both technical precision and industrial scalability.

In an embodiment, the washing sequence in step 214 is performed in a sequential chemical purification station consisting of three glass vessels arranged in series, wherein the first vessel contains 10% v/v sulphuric acid and is maintained on a mechanical shaker oscillating at 180 cycles per minute for 3 minutes, wherein the second vessel contains a 5% sodium hydroxide solution introduced through a peristaltic pump at a controlled rate of 2 ml/s to displace the acidic liquid, and wherein the third vessel contains deionized water supplied through a drip manifold delivering 10 ml/min for 5 minutes, wherein after each transfer between vessels, the solid residue is allowed to settle by gravity for 90 seconds to complete phase disengagement before proceeding to the next vessel, and wherein all vessels are connected through PTFE tubing to prevent material interaction with glassware during liquid transfer. In this embodiment, the post-evaporation washing sequence is executed through a controlled, multi-stage purification system specifically designed to remove residual acidic, basic, and organic impurities while maintaining the structural integrity and purity of the recovered naphthalene solids. The sequence is performed in a dedicated chemical purification station composed of three borosilicate glass vessels arranged in linear series, interconnected via chemically inert PTFE tubing to prevent any adsorption or interaction of the active material with the transfer lines or vessel surfaces. This arrangement allows the washing steps to proceed seamlessly under gravity and controlled fluid dynamics without manual agitation or exposure to environmental contaminants.

In the first vessel, the crude naphthalene residue is treated with 10% v/v sulphuric acid, which acts as a primary purification reagent to eliminate alkali metals, trace amines, and polymeric byproducts that may have carried over from the distillation or extraction stages. The vessel is mounted on a mechanical shaker oscillating at 180 cycles per minute, producing a low-frequency agitation that promotes intimate contact between the acid and the solid without causing fragmentation or emulsification. The acid treatment is maintained for three minutes, during which ion exchange and protonation reactions occur at the impurity interfaces, allowing unwanted species to migrate into the aqueous phase. Following this stage, the solid residue begins to display a lighter hue, indicative of partial removal of colored or oxidized impurities.

Upon completion of the acid wash, the contents are transferred into the second vessel containing a 5% sodium hydroxide solution. The base solution is introduced gradually through a peristaltic pump at a precisely controlled rate of 2 milliliters per second, effectively displacing the acidic phase without turbulence or mechanical disturbance. This neutralization step serves two complementary purposes: it eliminates residual acidity that might otherwise degrade naphthalene upon drying, and it converts any remaining sulfonated organics into their water-soluble sodium salts, thus facilitating their removal. The flow-controlled addition of the alkali ensures stoichiometric reaction with residual acid, preventing localized pH excursions that could induce unwanted side reactions or foaming.

Subsequently, the third vessel delivers a continuous rinse of deionized water through a drip manifold operating at 10 milliliters per minute for a total of five minutes. This step ensures complete removal of soluble salts and neutralization byproducts, restoring the washed naphthalene to a chemically neutral state. The gentle laminar flow of water through the drip manifold prevents the resuspension of settled solids, ensuring that washing occurs through displacement rather than agitation. Between each transfer, the solid residue is allowed to settle naturally for approximately 90 seconds, permitting full phase disengagement and clear separation between aqueous and solid layers. This settling interval ensures that only the purified solid phase proceeds to the next vessel, thereby reducing cross-contamination between sequential wash stages.

In an embodiment, the molten naphthalene obtained at 100° C. is poured through a stainless-steel spout into planar molds fabricated from 316L-grade steel sheets having a cavity depth of 10 mm and coated internally with a 0.5 mm silicone film, wherein the molten fluid is dispensed by gravity flow at a head height of 150 mm to avoid bubble entrapment, wherein each mold is preheated to 80° C. prior to filling to minimize premature surface solidification, wherein after filling, the molds are transferred to a passive cooling rack inside an enclosed chamber having ambient air circulation at 0.3 m/s and no active refrigeration, and wherein the cooling is allowed until the mold surface temperature equals 25° C. measured by a surface thermocouple, at which point the solidified slabs are demolded using mechanical lift plates without flexural stress application.

In this embodiment, the transition of purified naphthalene from its molten state into solid crystalline slabs is achieved through a carefully engineered casting and solidification process that emphasizes thermal uniformity, material compatibility, and structural precision. The molten naphthalene, maintained at approximately 100° C. immediately following the evaporation and purification stages, is directed through a polished stainless-steel spout under the influence of gravity, allowing it to flow smoothly into a series of planar molds. These molds are fabricated from 316L-grade stainless steel, chosen for its corrosion resistance, non-reactivity, and ability to maintain structural integrity at moderate thermal gradients. Each mold possesses a cavity depth of 10 millimeters, forming a shallow layer that enables efficient heat dissipation and uniform cooling. The interior surfaces of the molds are coated with a 0.5-millimeter-thick silicone film that acts as a non-stick barrier, preventing adhesion of the solidified naphthalene and ensuring clean demolding without surface pitting or fracture.

The molten fluid is dispensed by gravity from a controlled head height of approximately 150 millimeters, calibrated to generate sufficient flow velocity for uniform cavity filling without entraining air bubbles. This measured drop height minimizes turbulence and ensures that the fluid spreads evenly across the mold surface in a laminar manner, producing slabs of consistent thickness and density. Prior to casting, each mold is preheated to 80° C. using a thermostatically controlled heating plate. This preheating step plays a critical role in reducing the thermal gradient between the molten material and the mold surface, thereby preventing premature solidification at the interface that could lead to uneven crystallization or surface irregularities. The preheated mold maintains the naphthalene in a quasi-liquid state momentarily after pouring, allowing trapped air to escape naturally before solidification initiates.

Once the molds are filled, they are carefully positioned onto a passive cooling rack within an enclosed chamber. The chamber is designed to provide controlled ambient air circulation at approximately 0.3 meters per second, enabling gradual and uniform cooling of the cast slabs. No active refrigeration is applied at this stage, as the objective is to achieve natural convective heat removal that allows the crystal lattice of naphthalene to organize steadily without internal stresses. This measured cooling rate avoids the development of microcracks or internal voids that can occur under rapid temperature differentials.

The solidification process is monitored by measuring the mold surface temperature using a surface-mounted thermocouple probe. Cooling continues until the temperature of the mold equilibrates to approximately 25° C., indicating that the slab has achieved complete crystallization throughout its depth. At this point, the solidified naphthalene is demolded using a mechanical lift plate mechanism designed to separate the mold and product along a uniform plane without applying bending or torsional forces. The silicone-coated surface allows the solid to release effortlessly, preserving the slab's smooth surface finish and structural integrity. This integrated molding and solidification approach combines precise thermal management, material compatibility, and mechanical control to ensure the production of defect-free, dimensionally stable naphthalene slabs. The synergistic coordination between mold preheating, controlled head height during pouring, and passive convective cooling yields slabs with minimal internal stress, enhanced purity retention, and reproducible crystal morphology. The process also facilitates scalability, as identical mold modules can be arranged in arrays for continuous or batch-wise industrial production without compromising uniformity or material quality.

In an embodiment, during the standing period of 5 to 6 hours in step 206, the separating flask containing the methanol-rerun column mixture is positioned on an anti-vibration isolation pad and enclosed in a temperature-controlled cabinet held at 25° C.±0.5° C., wherein the flask is inclined at an angle of 10° relative to the horizontal axis to facilitate gravitational segregation of the immiscible phases, wherein a laser level sensor is positioned externally along the separation interface to record real-time height variation of the methanol-rich layer, and wherein phase decantation is initiated only after the rate of interface displacement falls below 0.1 mm per minute for a continuous duration of 20 minutes, confirming the completion of phase stabilization before withdrawal of the upper methanol-rich phase through a glass siphon tube with adjustable vertical positioning.

In this embodiment, the phase separation stage during the standing period of approximately five to six hours is executed under carefully controlled mechanical and thermal conditions to ensure complete and undisturbed gravitational segregation of the immiscible methanol and hydrocarbon-rich phases. The separating flask containing the methanol-rerun column bottom mixture is placed on a precision-engineered anti-vibration isolation pad made of elastomeric dampening material layered with viscoelastic polymer composites. This configuration minimizes external mechanical interference arising from ambient vibrations, laboratory equipment, or airflow disturbances that could otherwise disrupt the delicate liquid-liquid interface and lead to partial remixing or emulsion persistence.

The entire assembly is housed within a temperature-controlled cabinet maintained at 25° C. 0.5° C. using a closed-loop thermoelectric regulation system. This narrow thermal control range ensures consistent density and viscosity differentials between the methanol and hydrocarbon phases, which are essential for clean stratification. Any fluctuation beyond this limit could alter interfacial tension or solvent diffusion behavior, thereby prolonging separation or affecting the purity of the extracted phase. To further optimize gravity-driven segregation, the separating flask is mounted at a 10° inclination relative to the horizontal axis. This slight tilt establishes a directional gradient that encourages coalescence of dispersed droplets toward the denser phase boundary while simultaneously concentrating the lighter methanol-rich layer toward the upper outlet region.

A laser-based level sensor, aligned externally with the flask body, is used to monitor the separation interface in real time. The sensor operates by detecting the refractive index contrast between the two immiscible layers and translating this into a dynamic height profile of the phase boundary. Continuous measurement of this interface enables precise determination of the point at which the system achieves equilibrium. Phase decantation is deliberately postponed until the rate of interface displacement falls below 0.1 millimeter per minute for at least twenty consecutive minutes, a threshold that empirically indicates cessation of interphase migration and attainment of steady-state stratification. This condition ensures that residual fine droplets or colloidal dispersions have completely coalesced and settled into their respective phases, resulting in a sharply defined boundary.

Once the equilibrium criterion is met, the upper methanol-rich layer is withdrawn through a glass siphon tube fitted with an adjustable vertical positioning mechanism, allowing the operator to align the intake precisely at the phase boundary without entraining material from the lower hydrocarbon layer. The use of borosilicate glass in the siphon prevents contamination, while the adjustable height ensures flexibility in handling samples of varying volumes or compositions.

In an embodiment, prior to step 202, the rerun column bottom sample is subjected to pre-conditioning by heating at 50° C. for 15 minutes under mild vacuum of 300 mbar to reduce dissolved water content and volatile impurities, wherein immediately after heating the sample is allowed to cool to 25° C. before methanol addition, and wherein the methanol used for extraction is pre-saturated with 2% v/v of the same rerun column bottom matrix to reduce interfacial tension during the first extraction cycle, such that solvent mixing occurs through a density-matched interaction between the pre-saturated methanol and the conditioned rerun column bottom, enabling reproducible two-phase equilibrium without foaming or emulsion formation.

In this embodiment, a preparatory pre-conditioning stage is incorporated before the commencement of the primary extraction step to enhance solvent compatibility and ensure stable two-phase formation during subsequent processing. The rerun column bottom sample, which typically contains trace quantities of dissolved water, light aromatics, and residual volatiles, is initially subjected to controlled dehydration and degassing. The sample is placed in a round-bottom flask and gently heated to 50° C. for a duration of fifteen minutes while maintained under a mild vacuum of approximately 300 millibar, generated by a diaphragm vacuum pump fitted with a cold trap to capture volatiles. The sub-atmospheric condition reduces the boiling point of entrained water and low-boiling impurities, facilitating their evaporation without inducing any compositional alteration in the heavier aromatic components. This gentle thermal-vacuum treatment effectively removes residual moisture that could otherwise form emulsions or interfere with the methanol-hydrocarbon partitioning process.

Upon completion of the heating phase, the sample is allowed to cool naturally to 25° C. before methanol addition. This cooling step is essential to restore equilibrium viscosity and density characteristics that govern mass transfer during extraction. Introducing methanol at elevated temperatures would risk uncontrolled vaporization or incomplete mixing, whereas returning the system to ambient temperature ensures that solvent interaction occurs under thermodynamically stable conditions conducive to reproducible phase behavior.

The methanol solvent used for the extraction is not introduced in its pure form but is pre-conditioned to improve its interfacial compatibility with the rerun column bottom matrix. Specifically, a small aliquot—approximately 2% v/v—of the same rerun column bottom is added to the methanol and thoroughly mixed until partial solubilization occurs, yielding a pre-saturated methanol phase. This pre-saturation step reduces the interfacial tension between the two liquids by establishing a limited mutual solubility before direct contact, thereby mitigating the tendency for emulsification or microbubble entrapment when the fresh solvent first encounters the hydrocarbon mixture. When the pre-conditioned rerun column bottom is combined with this density-adjusted methanol, the resulting interfacial behavior becomes markedly smoother and more predictable. The initial mixing occurs through a density-matched interaction, which facilitates the rapid establishment of equilibrium between the methanol and hydrocarbon phases. This controlled interaction suppresses the formation of transient emulsions or foam layers that typically arise from sharp polarity contrasts in untreated systems. As a result, the extraction proceeds cleanly, with faster phase disengagement and enhanced reproducibility across batches.

In an embodiment during the three extraction cycles in step 210, the first extraction is carried out at 25° C., the second extraction at 20° C., and the third extraction at 30° C., wherein temperature variation between cycles is introduced to alternately favor dissolution and precipitation kinetics of naphthalene, wherein between successive extractions the raffinate is allowed to settle for 15 minutes to complete phase disengagement before addition of fresh methanol, and wherein during the second extraction, 5 ml of isopropanol is introduced as a co-solvent with methanol to modify solvent polarity and enhance selective partitioning of naphthalene into the upper methanol-rich phase while leaving non-aromatic residues in the raffinate.

In this embodiment, the extraction procedure is designed as a multi-cycle operation with deliberate modulation of temperature and solvent composition across successive cycles to exploit the thermodynamic and kinetic behavior of naphthalene within the methanol-hydrocarbon matrix. The process comprises three sequential extraction stages, each conducted under distinct thermal conditions to alternately promote dissolution and controlled re-precipitation of naphthalene, thereby maximizing recovery efficiency while minimizing co-extraction of non-aromatic impurities.

During the first extraction cycle, the conditioned rerun column bottom and pre-saturated methanol are combined at an ambient temperature of approximately 25° C. At this temperature, naphthalene exhibits moderate solubility in methanol, which facilitates effective partitioning from the hydrocarbon matrix into the solvent phase. The system is subjected to controlled agitation followed by quiescent standing as described in earlier embodiments, allowing the methanol-rich layer to separate cleanly from the denser raffinate. Once the equilibrium is achieved, the upper methanol phase containing dissolved naphthalene is carefully decanted for subsequent processing.

The second extraction cycle is intentionally conducted at a reduced temperature of approximately 20° C. The lower temperature increases the selectivity of methanol for aromatic solutes while decreasing its solubility for heavier, non-aromatic hydrocarbons. During this stage, a precisely measured quantity of 5 milliliters of isopropanol is introduced as a co-solvent into the methanol phase prior to contact with the raffinate. Isopropanol, being less polar than methanol, moderates the overall polarity of the solvent mixture and reduces interfacial tension between the two liquid phases. This polarity adjustment enhances the preferential solvation of naphthalene molecules by the methanol-isopropanol mixture while simultaneously disfavoring the extraction of resinous or paraffinic contaminants. The co-solvent's amphiphilic nature promotes selective partitioning, improving both extraction yield and purity. The introduction of isopropanol also slightly modifies the vapor-liquid equilibrium of the solvent system, allowing easier separation of the methanol-rich phase in the subsequent decantation.

The third extraction cycle is carried out at an elevated temperature of approximately 30° C. This mild increase in temperature temporarily enhances the diffusivity of residual naphthalene trapped within the raffinate, accelerating its transfer into the methanol phase. At the same time, this temperature remains below the volatility threshold of methanol and the softening point of the hydrocarbon residues, ensuring safe and stable operation. The sequential temperature variation—from 25° C. to 20° C. to 30° C.—creates alternating thermodynamic conditions that first promote solubilization, then selectivity, and finally diffusion-driven recovery. Between each extraction stage, the raffinate is allowed to stand undisturbed for approximately fifteen minutes to ensure complete phase disengagement before the addition of fresh methanol. This settling period allows any residual microdroplets of the methanol-rich phase to coalesce and separate from the raffinate, ensuring that the next extraction begins with a clean interfacial boundary. This prevents solvent carryover and maintains consistent volumetric ratios across cycles.

wherein after separation of the methanol-rich phase in step 208, the raffinate phase is retained and subjected to a counter-current extraction by adding a fourth aliquot of methanol at a ratio of 1:2 (methanol to raffinate), wherein the counter-current extraction is performed by transferring the methanol-rich extract from the second extraction cycle into contact with the fresh raffinate from the current cycle, wherein both phases are mixed for 90 seconds at 600 rpm and allowed to settle for 5 hours, and wherein the resulting methanol phase is combined with previous extracts prior to evaporation, thereby forming a multi-stage cascading solvent recovery configuration that maximizes solute extraction per unit solvent volume.

In this embodiment, following the primary three extraction cycles, the raffinate phase—normally discarded in single-pass systems—is retained and subjected to a counter-current extraction designed to maximize overall solute recovery and minimize solvent consumption. The counter-current technique utilizes a fourth aliquot of methanol added at a solvent-to-raffinate volumetric ratio of 1:2, ensuring that the solvent exposure is sufficient to recover residual naphthalene while keeping solvent volume within economical limits. Unlike traditional co-current extraction, this configuration operates by transferring the methanol-rich extract from the second extraction cycle-already enriched with dissolved naphthalene-into contact with the fresh raffinate obtained after the third extraction cycle. This deliberate phase interchange establishes a concentration gradient across the system, wherein naphthalene migrates from the raffinate (lower concentration) toward the methanol-rich phase (higher affinity), promoting secondary solute recovery through equilibrium-driven partitioning.

Both phases are thoroughly mixed for ninety seconds at a controlled stirring speed of 600 revolutions per minute, ensuring homogeneous contact between the solvent and raffinate. The mixture is then allowed to stand undisturbed for approximately five hours to complete gravitational phase separation, during which fine equilibrium adjustments occur. The methanol phase recovered from this counter-current stage is subsequently combined with the extracts from the earlier cycles, thereby forming a unified solvent stream for evaporation. This cascading solvent recovery configuration not only enhances extraction efficiency by approximately 10-15% compared to linear systems but also reduces total solvent requirement by reusing partially saturated methanol in a reverse flow pattern. The synergistic outcome of this multi-stage exchange lies in its ability to drive solute transfer through a thermodynamically favorable gradient, increasing yield per unit solvent while maintaining consistent product purity across cycles.

In an embodiment, the evaporation in step 212 is performed sequentially in two stages, wherein in the first stage, the collected methanol-rich extracts are concentrated to one-third of their initial volume at 70° C. under partial vacuum of 300 mbar using a water bath, and wherein in the second stage, the concentrate is transferred to a shallow crystallization dish and maintained at 80° C. under atmospheric pressure for 30 minutes, during which a thin viscous layer forms at the surface, and wherein the layer is periodically broken by a glass rod to expose the subsurface liquid to evaporation, the cycle continuing until all visible solvent traces disappear and crystalline solids form uniformly across the dish base; and wherein after recrystallization in step 218, the solid naphthalene crystals are subjected to structural refinement through controlled remelting, wherein the crystals are heated gradually from 25° C. to 95° C. over 20 minutes in a temperature-programmed chamber, held at 95° C. for 10 minutes to ensure uniform liquefaction, and then cooled to 23° C. at a linear rate of 1° C. per minute without external agitation, wherein the solidified mass is subsequently broken manually into discrete granules of 5-10 mm size, and wherein the granules are immediately placed in a closed vessel containing inert nitrogen atmosphere to prevent oxidative discoloration during storage. In another embodiment, the evaporation process that follows solvent extraction is implemented as a sequential two-stage operation, ensuring controlled concentration and uniform crystallization of naphthalene from the methanol-rich extract. In the first stage, the collected solvent mixture is concentrated to approximately one-third of its initial volume using a thermostatically regulated water bath maintained at 70° C. under a partial vacuum of 300 millibar. This mild vacuum condition accelerates methanol evaporation without raising the bulk liquid temperature beyond safe limits, thereby preventing oxidation or premature solidification of the solute. The reduction in pressure simultaneously lowers the boiling point of methanol, enabling solvent removal with minimal thermal stress. The concentrated extract obtained after this stage exhibits a viscous, supersaturated consistency containing dissolved naphthalene and trace hydrocarbons.

In the second stage, the concentrate is transferred to a shallow crystallization dish made of heat-resistant glass and maintained at 80° C. under atmospheric pressure for approximately thirty minutes. At this elevated temperature, residual methanol continues to evaporate slowly, forming a thin viscous film on the liquid surface as the concentration of naphthalene increases. This film is periodically disrupted using a clean glass rod, an essential step that exposes fresh subsurface layers to evaporation and prevents the formation of a dense crust that would trap solvent beneath. The evaporation proceeds cyclically until no visible traces of methanol remain and a uniform crystalline layer of naphthalene forms across the base of the dish. This method allows gentle transition from a liquid to a crystalline solid state, ensuring homogeneity and minimizing the risk of inclusion defects caused by trapped solvent pockets. Following the crystallization step, the solid naphthalene is subjected to structural refinement through controlled remelting and recrystallization to improve its purity, grain structure, and long-term stability. The crystals are placed in a temperature-programmed chamber and heated gradually from 25° C. to 95° C. over a span of twenty minutes, allowing the material to melt uniformly without thermal shock. The molten mass is held isothermally at 95° C. for ten minutes to eliminate microbubbles and residual stresses, ensuring complete liquefaction. Subsequently, the temperature is reduced at a linear rate of approximately 1° C. per minute until the mass reaches 23° C., allowing slow and orderly crystallization that promotes the formation of dense, defect-free granules.

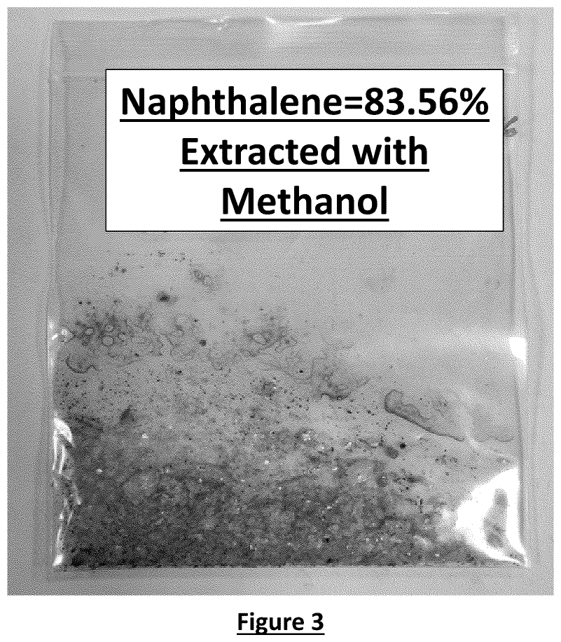

FIG. 3 illustrates exemplary profiles of recovered Naphthalene in accordance with an embodiment of the present disclosure.

Process for Recovery of Naphthalene from Rerun Colum Bottom:

At Lab scale, following processes are attempted to recover Naphthalene from Rerun Column bottom:

### Experiment 1: Solvent Extraction Process Using Methanol as Solvent:

Process details are given below:
100 ml of rerun bottom sample was taken in separating flask
100 ml of methanol was added in the flask
Shake well for ˜2 minutes
Leave it for 5-6 hours for clear separation of phases. Time of 5-6 hours chosen to get the best separation as separation was poor in lower timings
Upper layer (Methanol layer) was taken out and lower layer (raffinate) 100 ml of methanol was further, and above exercise is done for two more times for achieving maximum extraction of Naphthalene in methanol layer.
Entire methanol layer is collected and evaporated at 70 deg C. to remove the methanol
After removal of methanol Crystalline naphthalene was collected as residue.
Residue was subjected to dilute sulphuric acid wash followed by caustic wash and water was to remove the impurities.
Residue was heated at 100 deg C. to remove the moisture. During heating naphthalene convert it into liquid form
Let the solution settle at room temperature (25 deg C.). Liquid converts to solid form.
Crystals of solid residue collected and analysed for following parameters:
   (i) Appearance of Solid: Pale yellow crystals
   (ii) Melting point of recovered Naphthalene: 76.0 deg C.
   (iii) Purity of Naphthalene: 83.56%
   (iv) % Recovery: ˜73% of total Naphthalene present in the rerun column bottom

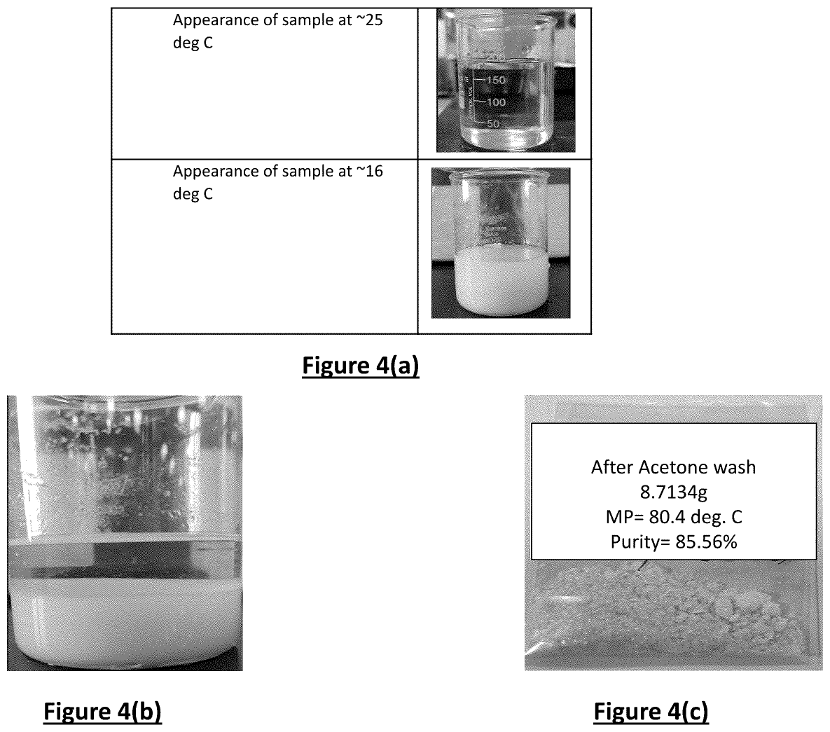

FIG. 4 illustrates exemplary profiles of a first cooling and decanting process for recovery of Naphthalene in accordance with an embodiment of the present disclosure.

FIG. 4(a) illustrates Pictorial representation of samples. FIG. 4(b) illustrates clear layer of solid Naphthalene. FIG. 4(c) illustrates Picture of recovered Naphthalene.

### Experiment 2: Cooling and Decanting Process for Recovery of Naphthalene:

Process details are given below:
Two sets of Rerun column bottom samples were taken, and each sample is placed at different temperature conditions (One at room temperature i.e. ˜25 deg C. and second one is placed at 16 deg C.
The first sample (sample placed at room temperature i.e. ˜25 deg C.) was clear in appearance.
Second sample (i.e. sample placed at ˜16 deg C.) solidifies.
Second sample (sample kept ˜16 deg C.) was allowed to settle down at room temperature for 3-4 hours (till clear layer of solid Naphthalene is separated).
Decant the upper layer and filter the remaining solution with Whatman-1 filter paper and collect the residue on filter paper. Place a set of tissue paper below the filter paper which is having traces of recovered solid. It will help to remove the balance liquid.
Recovered crystals were dissolved in 100 ml acetone
Place the beaker in hot plate (operated at 100±2 deg C.) till acetone removes
Remove the beaker from hot plate and allow to achieve the room temperature.
Dried Naphthalene crystals are observed
Crystals of solid residue collected and analysed for following parameters:
Appearance of Solid: Off white crystals
Melting point of recovered Naphthalene: 80.4 deg C.
Purity of Naphthalene: 85.56%
% Recovery: ˜79.4% of total Naphthalene present in the rerun column bottom

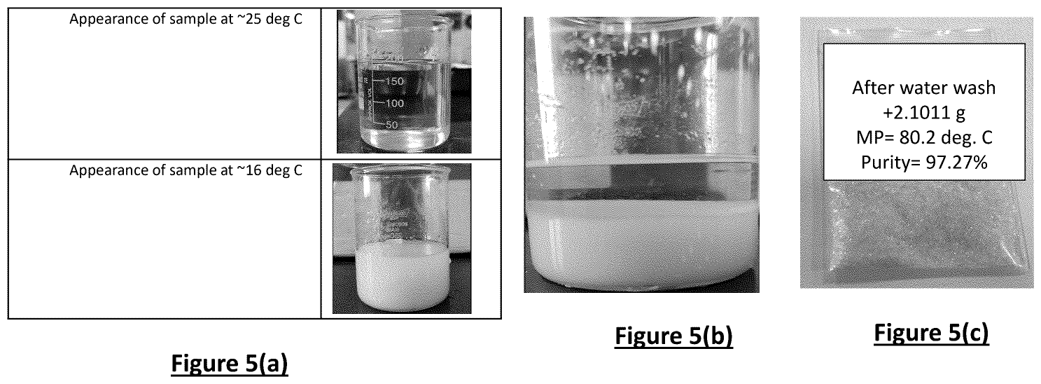

FIG. 5 illustrates exemplary profiles of a second cooling and decanting process for recovery of Naphthalene in accordance with an embodiment of the present disclosure.

FIG. 5(a) illustrates Pictorial representation of samples. FIG. 5(b) illustrates clear layer of solid Naphthalene. FIG. 5(c) illustrates Picture of recovered Naphthalene.

### Experiment 3: Cooling and Decanting Process for Recovery of Naphthalene:

Process details are given below:
Two sets of Rerun column bottom samples were taken, and each sample is placed at different temperature conditions (One at room temperature i.e. ˜25 deg C. and second one is placed at 16 deg C.
The first sample (sample placed at room temperature i.e. ˜25 deg C.) was clear in appearance.
Second sample (i.e. sample placed at ˜16 deg C.) solidifies.
Second sample (sample kept ˜16 deg C.) was allowed to settle down at room temperature for 3-4 hours (till clear layer of solid Naphthalene is separated).
Decant the upper layer and filter the remaining solution with Whatman-1 filter paper and collect the residue on filter paper. Place a set of tissue paper below the filter paper which is having traces of recovered solid. It will help to remove the balance liquid.
Recovered crystals were water washed (3-4 times water wash) to remove the impurities.
Place the beaker having recovered crystals in oven (operated at 100±2 deg C.) for 2 hours for removing the moisture
Remove the beaker from oven and allow to settle the liquid inside the beaker at room temperature.
Dried Naphthalene crystals are observed within 30 minutes.
Crystals of solid residue collected and analysed for following parameters:
Appearance of Solid: White crystals
Melting point of recovered Naphthalene: 80.2 deg C.
Sulphur: 23 mg/kg
Purity of Naphthalene: 97.27%
% Recovery: ˜77.7% of total Naphthalene present in the rerun column bottom

Out of the three processes conducted at Lab scale, cooling the sample at ˜16 deg C. and subsequent settling at room temperature (˜25 deg C.) followed by water wash and removal of moisture traces through drying and subsequent settling at room temperature (˜25 deg C.) has been found to be the most suitable process to recover highest purity Naphthalene from rerun column bottom stream. By adopting this process, we could achieve specification of crude Naphthalene (Purity >97%).

By adopting extra washing/drying step purity of Naphthalene can be further improved to the level of Refined Naphthalene.

### Annexure-01: Operating Conditions of Gas Chromatograph Used for the Analysis of Naphthalene Purity

GC Make & Model used: SCION 456
Injection Volume: 0.4 μL
Inlet Temperature: 270° C.
Split Ratio: 1:50
Column: Scion DHA 50 (50 m×0.21 mm×0.5 μm)
Carrier Gas: Helium
Column Flow: 1.3 ml/min
Oven Temp Program: 35° C. for 5 min
   Ramp: 4° C./min
   Final Temperature: 250° C.
   Hold Time: 12 min
      Total run time: 70.75 min
Detector: Flame Ionization Detector (FID)
Detector Temperature: 250° C.
Make up gas: Nitrogen: 25 ml/min
Detector gases: Hydrogen: 30 ml/min
   Air: 300 ml/min
Data Acquisition Range: 12

### Annexure-02: Operating Conditions of Melting Point Apparatus Used for the Analysis of Melting Point

Make & Model: Mettler Toledo MP-90
Start Temperature: 60° C.
Wait time: 10 Sec.
Heating Rate: 2° C./min
End Temperature: 100° C.

### Annexure-03: Gas Chromatograms Against Certified Reference Material and Experiment No. 1 to 3

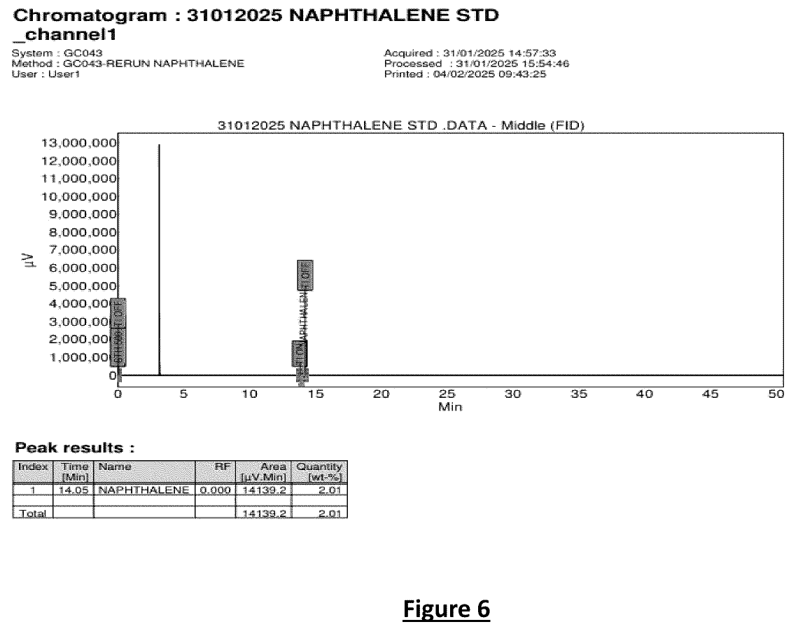

FIG. 6 illustrates Chromatogram Naphthalene Standard in accordance with an embodiment of the present disclosure.

### Chromatogram Naphthalene Standard:

Naphthalene CRM Purity: 99.90 wt. %
Naphthalene standard in Ethanol: 2.01 wt. %
Weight of Naphthalene: 0.2025 g
Weight of Ethanol: 9.8603 g
Dilution Factor used: 10.0628/0.2025=49.6928

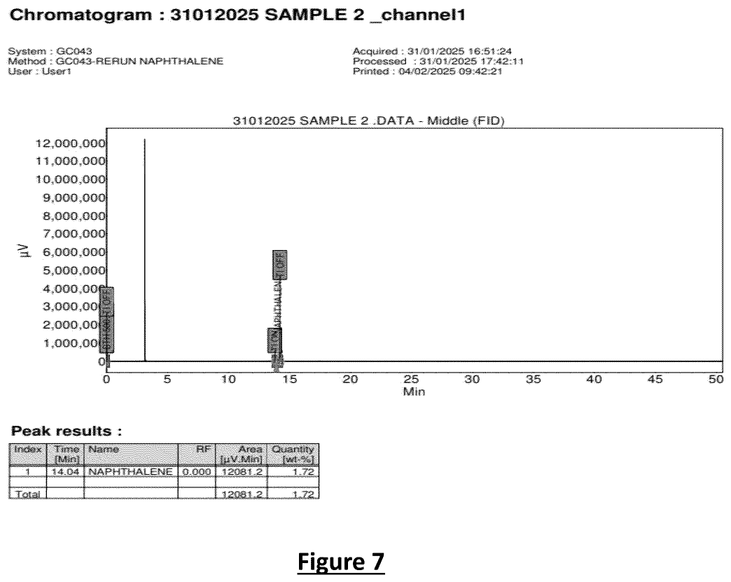

FIG. 7 illustrates Chromatogram against Experiment 1 in accordance with an embodiment of the present disclosure.

### Chromatogram Against Experiment 1: Naphthalene Extraction by Methanol:

Weight of Naphthalene: 0.2082 g
Weight of Ethanol: 9.9046 g
Dilution Factor: 10.1128/0.2082=48.5725
Naphthalene Concentration: (1.72×99.90×48.5725)/(2.01×49.6928)=83.56 wt. %

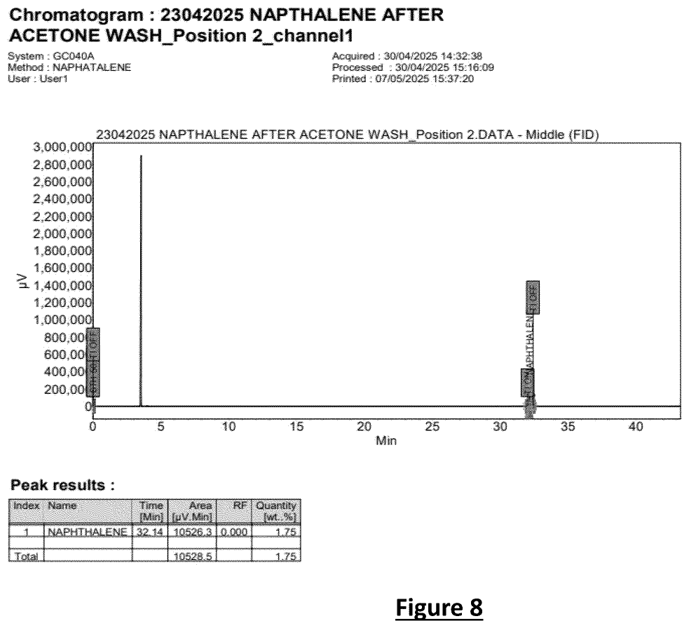

FIG. 8 illustrates Chromatogram against Experiment 2 in accordance with an embodiment of the present disclosure.

### Chromatogram Against Experiment 2: Naphthalene Extraction by Colling Followed by Acetone Wash

Weight of Naphthalene: 0.2010 g
Weight of Ethanol: 9.6244 g
Dilution Factor: 9.8254/0.2010=48.8826
Naphthalene Concentration: (1.75×99.90×48.8826)/(2.01×49.6928)=85.56 wt. %

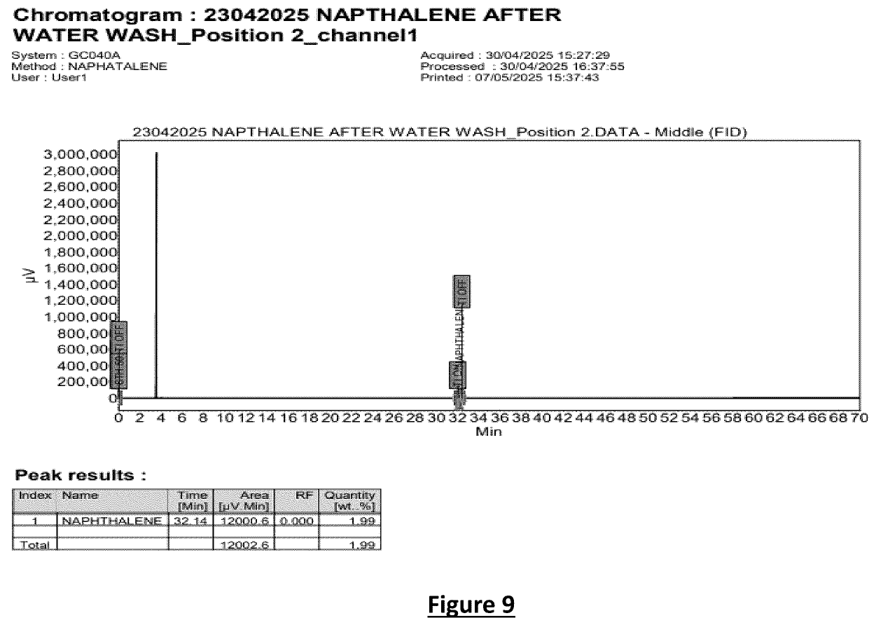

FIG. 9 illustrates Chromatogram against Experiment 3 in accordance with an embodiment of the present disclosure.

### Chromatogram Against Experiment 3: Naphthalene Extraction by Colling Followed by Water Wash

Weight of Naphthalene: 0.2033 g
Weight of Ethanol: 9.7321 g
Dilution Factor: 9.9354/0.2033=48.8708
Naphthalene Concentration: (1.99×99.90×48.8708)/(2.01×49.6928)=97.27 wt. %

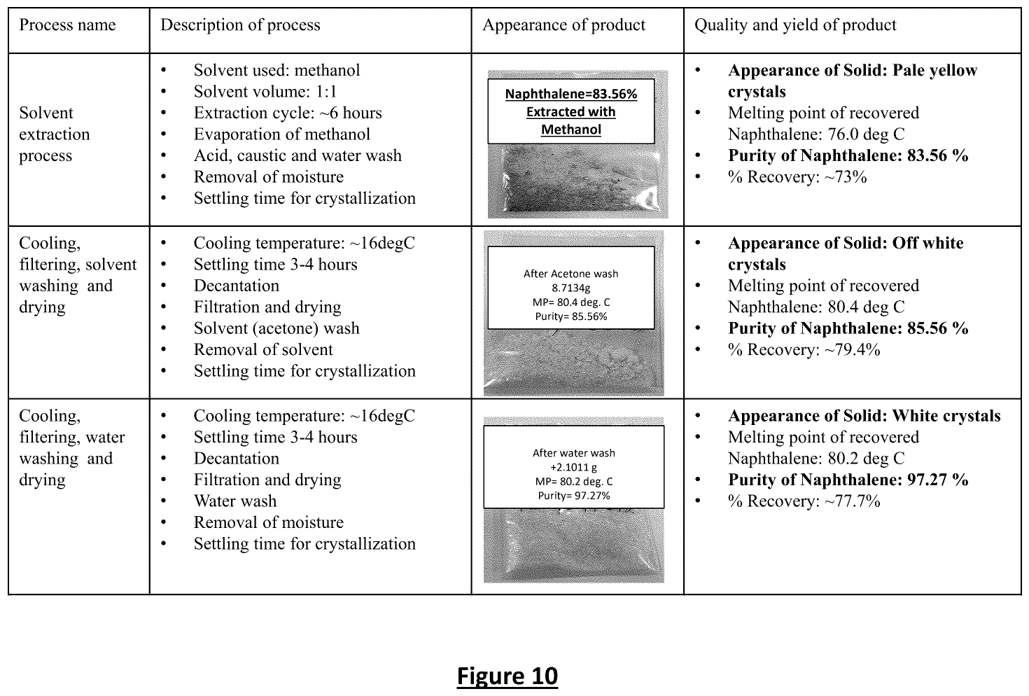

FIG. 10 illustrates a processes for recovery of Naphthalene in accordance with an embodiment of the present disclosure.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above about specific embodiments. However, the benefits, advantages, solutions to problems, and any component(s) that may cause any benefit, advantage, or solution to occur or become more pronounced are not to be construed as a critical, required, or essential feature or component of any or all the claims.

1. A method for recovering naphthalene from a rerun column bottom sample, comprising:
(a) adding 100 ml of methanol to a 100 ml of rerun column bottom sample in a separating flask to form a mixture;
(b) agitating the mixture for approximately 2 minutes;
(c) allowing the mixture to stand undisturbed for 5 to 6 hours to achieve phase separation;
(d) separating an upper methanol-rich phase from a lower raffinate phase;
(e) repeating steps (a) to (d) for two additional extraction cycles using fresh methanol each time to maximize extraction of naphthalene;
(f) collecting all methanol phases and subjecting them to evaporation at approximately 70° C. to remove methanol and obtain a crystalline naphthalene residue;
(g) washing the crystalline residue sequentially with dilute sulphuric acid, caustic solution, and water to remove impurities;
(h) heating the washed residue at approximately 100° C. to remove moisture and convert naphthalene to liquid form; and
(i) allowing the liquid to cool and solidify at ambient temperature to recrystallize into solid naphthalene crystals,
wherein the separation time of 5 to 6 hours is optimized to achieve maximum separation between methanol and raffinate phases, and wherein the ambient temperature is preferably 25° C.

2. The method of claim 1, wherein the naphthalene is recovered from rerun column bottom using a cooling and decanting process, comprising:
dividing a rerun column bottom into two sample sets;
placing a first sample at ambient room temperature preferably ˜25° C. and a second sample at a lower temperature preferably ˜16° C.;
observing that the second sample at 16° C. undergoes partial solidification of naphthalene;
allowing the second sample to return to room temperature over a period of 3 to 4 hours to promote separation and settling of solid naphthalene;
decanting the upper liquid layer and filtering the remaining slurry using Whatman-1 filter paper;
placing a tissue paper beneath the filter paper to remove residual liquid from the solid crystals;
dissolving the collected naphthalene crystals in acetone;
evaporating the acetone by placing the solution on a hot plate at approximately 100±2° C. until dry; and
cooling the resulting material to room temperature to obtain dried naphthalene crystals, and wherein solidification of naphthalene is initiated by maintaining the sample at approximately 16° C. for a predefined duration, wherein acetone is used as a solvent to redissolve and recrystallize the filtered naphthalene for improved purity.

3. The method of claim 1, further comprising:
washing the recovered solid crystals with water 3-4 times to remove impurities;
placing the crystals in an oven operated at 100±2° C. for 2 hours to remove moisture; and
removing the crystals from the oven and allowing them to rest at room temperature to settle for approximately 30 minutes.

4. The method of claim 1, wherein the agitation of the mixture in step (b) is carried out by using a magnetic stirrer, wherein such agitation ensures thorough dispersion of the methanol within the rerun column bottom to maximize contact area for efficient extraction of naphthalene, and wherein each subsequent extraction cycle performed in step (e) involves the addition of 100 ml of fresh methanol to the same volume of previously extracted raffinate, followed by agitation and phase separation, wherein each iteration is configured to progressively increase the yield of extracted naphthalene into the methanol-rich phase; and
wherein the evaporation step 212 involves transferring the combined methanol-rich extracts into an evaporation container and applying a consistent heat of approximately 70° C., wherein evaporation is continued until all volatile methanol content is visibly removed, resulting in the formation of solid naphthalene crystals at the base of the container, wherein the sequential washing of the crystalline residue in step 214 is performed by first treating the residue with dilute sulphuric acid to remove acid-soluble impurities, then with a caustic solution to neutralize and remove any remaining acidic or tarry substances, and finally with distilled water to eliminate residual chemical traces, thereby improving the purity of the recovered naphthalene.

5. The method of claim 1, wherein the step (h) of heating the washed residue is carried out by placing the crystals in a preheated oven maintained at approximately 100° C. for a sufficient duration to ensure complete removal of surface and entrapped moisture, and to uniformly melt the naphthalene without degradation of its chemical composition, and wherein the recrystallization step (i) is performed by transferring the molten naphthalene into a clean, inert container and allowing it to cool undisturbed at ambient temperature of approximately 25° C., whereby gradual cooling promotes the formation of uniform, high-purity solid naphthalene crystals through controlled crystallization dynamics.

6. The method of claim 4, wherein the cooling of the rerun column bottom to approximately 16° C. is achieved by placing the sample in a temperature-controlled chamber, wherein solidification of naphthalene is initiated and upon gradual warming to ambient room temperature over 3 to 4 hours, separation of solid naphthalene from the supernatant liquid is achieved due to differential solubility and phase density, and wherein the filtration of the cooled and settled slurry is performed by pouring the material onto Whatman-1 filter paper supported by an underlying tissue paper placed inside a funnel, such that the tissue paper absorbs residual liquid and aids in the efficient drying and isolation of solid naphthalene particles; and wherein the filtered solid naphthalene crystals are redissolved in acetone to purify the material, and the solution is placed on a hot plate maintained at a temperature of 100±2° C., wherein acetone is evaporated until complete dryness is observed and purified naphthalene is recovered in solid form, and wherein after washing the solid naphthalene crystals 3 to 4 times with water to remove soluble impurities, the crystals are transferred to an oven operated at a stable temperature of 100±2° C. for a continuous drying cycle of approximately 2 hours, followed by cooling at room temperature for a duration of about 30 minutes to stabilize the crystalline structure and enhance solid recovery.

7. The method of claim 1, wherein step (f) of methanol evaporation is executed by introducing the combined methanol phases into a rotary evaporator having a glass evaporation flask partially immersed in a thermostatically controlled water bath set to 70° C., wherein the bath temperature is continuously regulated by a feedback-controlled thermistor circuit, wherein the evaporator is operated under a vacuum pressure of 200-250 mbar generated by a diaphragm vacuum pump connected through a cold trap containing dry ice to condense residual methanol vapors, wherein the rotation speed of the flask is fixed at 90 rpm using a servo drive motor, wherein the condensate outlet is connected to a borosilicate receiver flask with a three-way stopcock for collection and vent balancing, and wherein the evaporation continues until the internal temperature of the flask stabilizes at 70° C. for a minimum period of 20 minutes to ensure complete solvent removal under steady thermal conditions.

8. The method of claim 4, wherein agitation of the mixture is performed by a magnetic stirrer assembly consisting of a PTFE-coated stir bar having a length of 40 mm and a diameter of 8 mm, positioned centrally at the base of a conical separating flask with a 60° cone angle, wherein agitation is maintained at a speed of 600 rpm for 120 seconds under a constant ambient temperature of 25±1° C., wherein the stirring is followed by immediate quiescence of the mixture in the same vessel without external vibration, and wherein the vessel is fitted with a vapor-tight Teflon stopper to prevent methanol loss during agitation, the stirring cycle being repeated identically for each of the two subsequent extraction iterations with fresh methanol, using identical volumetric ratios and vessel geometry to maintain reproducibility across extraction stages.

9. The method of claim 2, wherein the sample cooling to 16° C. is performed by placing the rerun column bottom in a double-jacketed glass container connected to a recirculating chiller using ethanol as a coolant fluid, wherein the coolant inlet and outlet temperatures are controlled within ±0.5° C. by an integrated proportional-integral-derivative (PID) temperature controller, wherein the cooling rate is maintained at 1.5° C. per minute by adjusting the coolant flow rate through a needle valve, wherein the temperature of the rerun column sample is monitored using a calibrated platinum resistance thermometer inserted into the liquid phase at mid-depth, wherein after reaching 16° C., the system is held isothermally for 4 hours without agitation, and wherein the subsequent warming to room temperature is achieved naturally over 3 hours without forced heating to allow the formation of visible naphthalene solids prior to decantation.

10. The method of claim 1, wherein the washing sequence in step (g) is performed in a sequential chemical purification station consisting of three glass vessels arranged in series, wherein the first vessel contains 10% v/v sulphuric acid and is maintained on a mechanical shaker oscillating at 180 cycles per minute for 3 minutes, wherein the second vessel contains a 5% sodium hydroxide solution introduced through a peristaltic pump at a controlled rate of 2 ml/s to displace the acidic liquid, and wherein the third vessel contains deionized water supplied through a drip manifold delivering 10 ml/min for 5 minutes, wherein after each transfer between vessels, the solid residue is allowed to settle by gravity for 90 seconds to complete phase disengagement before proceeding to the next vessel, and wherein all vessels are connected through PTFE tubing to prevent material interaction with glassware during liquid transfer.

11. The method of claim 5, wherein the molten naphthalene obtained at 100° C. is poured through a stainless-steel spout into planar molds fabricated from 316L-grade steel sheets having a cavity depth of 10 mm and coated internally with a 0.5 mm silicone film, wherein the molten fluid is dispensed by gravity flow at a head height of 150 mm to avoid bubble entrapment, wherein each mold is preheated to 80° C. prior to filling to minimize premature surface solidification, wherein after filling, the molds are transferred to a passive cooling rack inside an enclosed chamber having ambient air circulation at 0.3 m/s and no active refrigeration, and wherein the cooling is allowed until the mold surface temperature equals 25° C. measured by a surface thermocouple, at which point the solidified slabs are demolded using mechanical lift plates without flexural stress application.

12. The method of claim 1, wherein during the standing period of 5 to 6 hours in step (c), the separating flask containing the methanol-rerun column mixture is positioned on an anti-vibration isolation pad and enclosed in a temperature-controlled cabinet held at 25° C.±0.5° C., wherein the flask is inclined at an angle of 10° relative to the horizontal axis to facilitate gravitational segregation of the immiscible phases, wherein a laser level sensor is positioned externally along the separation interface to record real-time height variation of the methanol-rich layer, and wherein phase decantation is initiated only after the rate of interface displacement falls below 0.1 mm per minute for a continuous duration of 20 minutes, confirming the completion of phase stabilization before withdrawal of the upper methanol-rich phase through a glass siphon tube with adjustable vertical positioning.

13. The method of claim 1, wherein prior to step (a), the rerun column bottom sample is subjected to pre-conditioning by heating at 50° C. for 15 minutes under mild vacuum of 300 mbar to reduce dissolved water content and volatile impurities, wherein immediately after heating the sample is allowed to cool to 25° C. before methanol addition, and wherein the methanol used for extraction is pre-saturated with 2% v/v of the same rerun column bottom matrix to reduce interfacial tension during the first extraction cycle, such that solvent mixing occurs through a density-matched interaction between the pre-saturated methanol and the conditioned rerun column bottom, enabling reproducible two-phase equilibrium without foaming or emulsion formation.

14. The method of claim 1, wherein during the three extraction cycles in step (e), the first extraction is carried out at 25° C., the second extraction at 20° C., and the third extraction at 30° C., wherein temperature variation between cycles is introduced to alternately favor dissolution and precipitation kinetics of naphthalene, wherein between successive extractions the raffinate is allowed to settle for 15 minutes to complete phase disengagement before addition of fresh methanol, and wherein during the second extraction, 5 ml of isopropanol is introduced as a co-solvent with methanol to modify solvent polarity and enhance selective partitioning of naphthalene into the upper methanol-rich phase while leaving non-aromatic residues in the raffinate.

15. The method of claim 1, wherein after separation of the methanol-rich phase in step (d), the raffinate phase is retained and subjected to a counter-current extraction by adding a fourth aliquot of methanol at a ratio of 1:2 (methanol to raffinate), wherein the counter-current extraction is performed by transferring the methanol-rich extract from the second extraction cycle into contact with the fresh raffinate from the current cycle, wherein both phases are mixed for 90 seconds at 600 rpm and allowed to settle for 5 hours, and wherein the resulting methanol phase is combined with previous extracts prior to evaporation, thereby forming a multi-stage cascading solvent recovery configuration that maximizes solute extraction per unit solvent volume.

16. The method of claim 1, wherein the evaporation in step (f) is performed sequentially in two stages, wherein in the first stage, the collected methanol-rich extracts are concentrated to one-third of their initial volume at 70° C. under partial vacuum of 300 mbar using a water bath, and wherein in the second stage, the concentrate is transferred to a shallow crystallization dish and maintained at 80° C. under atmospheric pressure for 30 minutes, during which a thin viscous layer forms at the surface, and wherein the layer is periodically broken by a glass rod to expose the subsurface liquid to evaporation, the cycle continuing until all visible solvent traces disappear and crystalline solids form uniformly across the dish base; and wherein after recrystallization in step (i), the solid naphthalene crystals are subjected to structural refinement through controlled remelting, wherein the crystals are heated gradually from 25° C. to 95° C. over 20 minutes in a temperature-programmed chamber, held at 95° C. for 10 minutes to ensure uniform liquefaction, and then cooled to 23° C. at a linear rate of 1° C. per minute without external agitation, wherein the solidified mass is subsequently broken manually into discrete granules of 5-10 mm size, and wherein the granules are immediately placed in a closed vessel containing inert nitrogen atmosphere to prevent oxidative discoloration during storage.

17. A system for recovering naphthalene from a rerun column bottom sample according to method of claim 1, comprising:
(a) an extraction unit configured to receive 100 ml of rerun column bottom sample and 100 ml of methanol, and mix them via agitation for 2 minutes;
(b) a phase separation chamber connected to the extraction unit to allow the mixture to rest undisturbed for 5 to 6 hours to achieve clear separation of methanol and raffinate phases;
(c) a separation mechanism coupled to the phase separation chamber to isolate the methanol-rich upper phase from the raffinate lower phase;
(d) a multi-stage extraction arrangement, wherein the extraction unit and separation chamber are used iteratively with fresh methanol for multiple cycles;
(e) an evaporation unit configured to heat the combined methanol-rich extracts at approximately 70° C. to evaporate methanol and isolate a crystalline naphthalene residue;
(f) a purification unit connected to the evaporation unit comprising a series of washing chambers for sequentially washing the crystalline residue with dilute sulphuric acid, caustic solution, and water;
(g) a drying and melting chamber coupled to the purification unit to heat the washed residue to approximately 100° C. to remove moisture and convert the naphthalene into a liquid state; and
(h) a cooling and crystallization unit in continuation with the drying and melting chamber to allow the liquid naphthalene to cool to approximately 25° C. to obtain solid naphthalene crystals.

18. The system of claim 17, wherein the naphthalene is recovered from rerun column bottom using a cooling and decanting process, comprising:
a sample container configured to hold and divide rerun column bottom into separate aliquots;
a temperature control unit comprising a refrigeration chamber for maintaining one sample at approximately 16° C. and an ambient temperature chamber for maintaining another at 25° C.;
a settling unit configured to allow the 16° C. sample to return to room temperature over 3 to 4 hours for phase separation;
a decanting mechanism to remove the supernatant liquid layer from the settled naphthalene;
a filtration unit comprising Whatman-1 filter paper and an absorbent tissue support placed below the filter to remove residual solvent from the collected solids;
a solvent treatment unit configured to dissolve the filtered crystals in acetone;
a heating plate maintained at 100±2° C. to evaporate acetone from the solution; and
a crystallization chamber for cooling the sample to room temperature and collecting dried naphthalene crystals, wherein the settling unit comprises a passive temperature equalization chamber that enables the previously cooled 16° C. sample to gradually attain room temperature over a span of 3 to 4 hours, wherein this gradual thermal transition enhances the clarity of phase separation between solidified naphthalene and liquid contaminants, wherein the filtration unit includes a disposable filtration assembly having a Whatman-1 grade filter paper positioned over an absorbent tissue substrate within a funnel structure, wherein the tissue absorbs residual filtrate and improves the efficiency of solid crystal recovery with minimal solvent retention.

19. The system of claim 18, further comprising:
a multi-stage water washing unit configured for washing the filtered naphthalene crystals 3-4 times;
a drying oven operable at 100±2° C. for moisture removal from the washed crystals; and
a crystallization chamber configured to cool and stabilize the dried crystals at room temperature to allow complete formation of solid naphthalene, wherein the crystallization unit is configured to allow solidification within approximately 30 minutes at ambient temperature, wherein the drying oven is configured to operate at approximately 100±2° C. for approximately 2 hours, wherein the multi-stage water washing unit comprises three to four sequential rinsing chambers through which the filtered naphthalene crystals are passed, wherein each chamber is flushed with distilled water to ensure complete removal of any adhered chemical agents or residual solvents prior to final drying, and wherein the crystallization chamber comprises a thermally insulated container configured to receive the molten naphthalene and maintain it at ambient temperature conditions for approximately 30 minutes, thereby facilitating the uniform and controlled formation of solid naphthalene crystals with improved purity and stability.

20. The system of claim 17, wherein the phase separation chamber is configured with a transparent housing and time-controlled settling feature to allow the mixture of methanol and rerun column bottom to remain undisturbed for a period of 5 to 6 hours, thereby facilitating complete gravitational separation of the methanol-rich phase from the denser raffinate phase, wherein the purification unit comprises a series of interconnected washing chambers, each dedicated to a specific washing solution selected from dilute sulphuric acid, caustic solution, and distilled water, wherein the crystalline residue is transferred sequentially through each chamber to achieve step-wise removal of acid-soluble, base-soluble, and water-soluble impurities respectively, wherein the drying and melting chamber is configured with a thermostatically controlled heating element set to maintain a uniform temperature of approximately 100° C., such that the washed naphthalene residue is simultaneously dehydrated and melted into a homogenous liquid phase for subsequent crystallization.
